(19) United States
(12) Patent Application Publication
(10) Pub. No.: US 2026/0069204 A1
(43) Pub. Date: Mar. 12, 2026

(54) USER INTERFACES FOR RESPIRATORY DISTURBANCES

(71) Applicant: Apple Inc., Cupertino, CA (US)

(72) Inventors: Matthew W. CROWLEY, Sunnyvale, CA (US); Nicholas D. FELTON, Sunnyvale, CA (US); Edgar FRAUSTO, Santa Clara, CA (US)

(21) Appl. No.: 19/019,198

(22) Filed: Jan. 13, 2025

Related U.S. Application Data
(60) Provisional application No. 63/692,136, filed on Sep. 8, 2024.

(51) Int. Cl. A61B 5/00; A61B 5/113; G16H 40/67

(52) U.S. Cl. CPC A61B 5/4818; A61B 5/113; A61B 5/7435; A61B 5/7475; G16H 40/67; A61B 2562/0219

(57) ABSTRACT

The present disclosure generally relates to displaying respiratory disturbances in user interfaces.

### CROSS-REFERENCE TO RELATED APPLICATIONS

This application claims priority from U.S. Provisional Ser. No. 63/692,136, entitled “USER INTERFACES FOR RESPIRATORY DISTURBANCES”, filed on Sep. 8, 2024, which is hereby incorporated by reference in its entirety for all purposes.

### FIELD

The present disclosure relates generally to computer user interfaces, and more specifically to techniques for displaying respiratory disturbances.

### BACKGROUND

Wearable computer systems optionally measure various measurements and/or biometrics of a person's body. This measured information is optionally displayed by the wearable computer system and/or a computer system that is different from the wearable computer system.

### BRIEF SUMMARY

Some techniques for displaying respiratory disturbances using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for displaying respiratory disturbances. Such methods and interfaces optionally complement or replace other methods for displaying respiratory disturbances. Such methods and interfaces reduce the cognitive burden on a user and produce a more efficient human-machine interface. For battery-operated computing devices, such methods and interfaces conserve power and increase the time between battery charges.

In accordance with some embodiments, a method performed at a computer system that is in communication with a display generation component and one or more input devices is described. The method comprises: detecting, via the one or more input devices, a request to display a respiratory information user interface; and in response to detecting the request to display the respiratory information user interface, displaying, via the display generation component, the respiratory information user interface, wherein displaying the respiratory information user interface concurrently includes: displaying, via the display generation component, a graph of respiratory information, wherein the graph includes: one or more respiratory disturbances; and a plurality of time periods; in accordance with a determination that a first set of one or more respiratory disturbances satisfy a set of criteria during a first time period of the plurality of time periods, displaying, via the display generation component, a first graphical object at a first location, wherein the first location corresponds to the first time period; and in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

In accordance with some embodiments, a non-transitory computer-readable storage medium is described. The non-transitory computer-readable storage medium stores one or more programs configured to be executed by one or more processors of a computer system that is in communication with a display generation component and one or more input devices, the one or more programs including instructions for: detecting, via the one or more input devices, a request to display a respiratory information user interface; and in response to detecting the request to display the respiratory information user interface, displaying, via the display generation component, the respiratory information user interface, wherein displaying the respiratory information user interface concurrently includes: displaying, via the display generation component, a graph of respiratory information, wherein the graph includes: one or more respiratory disturbances; and a plurality of time periods; in accordance with a determination that a first set of one or more respiratory disturbances satisfy a set of criteria during a first time period of the plurality of time periods, displaying, via the display generation component, a first graphical object at a first location, wherein the first location corresponds to the first time period; and in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

In accordance with some embodiments, a transitory computer-readable storage medium is described. The transitory computer-readable storage medium stores one or more programs configured to be executed by one or more processors of a computer system that is configured to communicate with a display generation component and one or more input devices, the one or more programs including instructions for: detecting, via the one or more input devices, a request to display a respiratory information user interface; and in response to detecting the request to display the respiratory information user interface, displaying, via the display generation component, the respiratory information user interface, wherein displaying the respiratory information user interface concurrently includes: displaying, via the display generation component, a graph of respiratory information, wherein the graph includes: one or more respiratory disturbances; and a plurality of time periods; in accordance with a determination that a first set of one or more respiratory disturbances satisfy a set of criteria during a first time period of the plurality of time periods, displaying, via the display generation component, a first graphical object at a first location, wherein the first location corresponds to the first time period; and in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

In accordance with some embodiments, a computer system that is configured to communicate with a display generation component and one or more input devices is described. The computer system comprises: one or more processors; and memory storing one or more programs configured to be executed by the one or more processors, the one or more programs including instructions for: detecting, via the one or more input devices, a request to display a respiratory information user interface; and in response to detecting the request to display the respiratory information user interface, displaying, via the display generation component, the respiratory information user interface, wherein displaying the respiratory information user interface concurrently includes: displaying, via the display generation component, a graph of respiratory information, wherein the graph includes: one or more respiratory disturbances; and a plurality of time periods; in accordance with a determination that a first set of one or more respiratory disturbances satisfy a set of criteria during a first time period of the plurality of time periods, displaying, via the display generation component, a first graphical object at a first location, wherein the first location corresponds to the first time period; and in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

In accordance with some embodiments, a computer system that is configured to communicate with a display generation component and one or more input devices is described. The computer system comprises: means for detecting, via the one or more input devices, a request to display a respiratory information user interface; and means for, in response to detecting the request to display the respiratory information user interface, displaying, via the display generation component, the respiratory information user interface, wherein displaying the respiratory information user interface concurrently includes: displaying, via the display generation component, a graph of respiratory information, wherein the graph includes: one or more respiratory disturbances; and a plurality of time periods; in accordance with a determination that a first set of one or more respiratory disturbances satisfy a set of criteria during a first time period of the plurality of time periods, displaying, via the display generation component, a first graphical object at a first location, wherein the first location corresponds to the first time period; and in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

In accordance with some embodiments, a computer program product is described. The computer program product comprises one or more programs configured to be executed by one or more processors of a computer system that is that is in communication with a display generation component and one or more input devices. The one or more programs include instructions for: detecting, via the one or more input devices, a request to display a respiratory information user interface; and in response to detecting the request to display the respiratory information user interface, displaying, via the display generation component, the respiratory information user interface, wherein displaying the respiratory information user interface concurrently includes: displaying, via the display generation component, a graph of respiratory information, wherein the graph includes: one or more respiratory disturbances; and a plurality of time periods; in accordance with a determination that a first set of one or more respiratory disturbances satisfy a set of criteria during a first time period of the plurality of time periods, displaying, via the display generation component, a first graphical object at a first location, wherein the first location corresponds to the first time period; and in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

In accordance with some embodiments, a method performed at a computer system that is in communication with one or more display generation components is described. The method comprises: displaying, via the one or more display generation components, a respective user interface; and while displaying the respective user interface: in accordance with a determination that a set of respiratory disturbance criteria is satisfied over a period of time, displaying, via the one or more display generation components, a notification corresponding to one or more respiratory disturbances, wherein the set of respiratory disturbance criteria includes a set of sleep session criteria that is satisfied when at least a threshold amount of sleep sessions from a set of sleep sessions have at least a threshold amount of respiratory disturbances; and in accordance with a determination that the set of respiratory disturbance criteria is not satisfied for the period of time, forgoing display of the notification corresponding to the one or more respiratory disturbances.

In accordance with some embodiments, a non-transitory computer-readable storage medium is described. The non-transitory computer-readable storage medium stores one or more programs configured to be executed by one or more processors of a computer system that is in communication with one or more display generation components, the one or more programs including instructions for: displaying, via the one or more display generation components, a respective user interface; and while displaying the respective user interface: in accordance with a determination that a set of respiratory disturbance criteria is satisfied over a period of time, displaying, via the one or more display generation components, a notification corresponding to one or more respiratory disturbances, wherein the set of respiratory disturbance criteria includes a set of sleep session criteria that is satisfied when at least a threshold amount of sleep sessions from a set of sleep sessions have at least a threshold amount of respiratory disturbances; and in accordance with a determination that the set of respiratory disturbance criteria is not satisfied for the period of time, forgoing display of the notification corresponding to the one or more respiratory disturbances.

In accordance with some embodiments, a transitory computer-readable storage medium is described. The transitory computer-readable storage medium stores one or more programs configured to be executed by one or more processors of a computer system that is configured to communicate with one or more display generation components, the one or more programs including instructions for: displaying, via the one or more display generation components, a respective user interface; and while displaying the respective user interface: in accordance with a determination that a set of respiratory disturbance criteria is satisfied over a period of time, displaying, via the one or more display generation components, a notification corresponding to one or more respiratory disturbances, wherein the set of respiratory disturbance criteria includes a set of sleep session criteria that is satisfied when at least a threshold amount of sleep sessions from a set of sleep sessions have at least a threshold amount of respiratory disturbances; and in accordance with a determination that the set of respiratory disturbance criteria is not satisfied for the period of time, forgoing display of the notification corresponding to the one or more respiratory disturbances.

In accordance with some embodiments, a computer system that is configured to communicate with one or more display generation components is described. The computer system comprises: one or more processors; and memory storing one or more programs configured to be executed by the one or more processors, the one or more programs including instructions for: displaying, via the one or more display generation components, a respective user interface; and while displaying the respective user interface: in accordance with a determination that a set of respiratory disturbance criteria is satisfied over a period of time, displaying, via the one or more display generation components, a notification corresponding to one or more respiratory disturbances, wherein the set of respiratory disturbance criteria includes a set of sleep session criteria that is satisfied when at least a threshold amount of sleep sessions from a set of sleep sessions have at least a threshold amount of respiratory disturbances; and in accordance with a determination that the set of respiratory disturbance criteria is not satisfied for the period of time, forgoing display of the notification corresponding to the one or more respiratory disturbances.

In accordance with some embodiments, a computer system that is configured to communicate with one or more display generation components is described. The computer system comprises: means displaying, via the one or more display generation components, a respective user interface; and means for, while displaying the respective user interface: in accordance with a determination that a set of respiratory disturbance criteria is satisfied over a period of time, displaying, via the one or more display generation components, a notification corresponding to one or more respiratory disturbances, wherein the set of respiratory disturbance criteria includes a set of sleep session criteria that is satisfied when at least a threshold amount of sleep sessions from a set of sleep sessions have at least a threshold amount of respiratory disturbances; and in accordance with a determination that the set of respiratory disturbance criteria is not satisfied for the period of time, forgoing display of the notification corresponding to the one or more respiratory disturbances.

In accordance with some embodiments, a computer program product is described. The computer program product comprises one or more programs configured to be executed by one or more processors of a computer system that is that is in communication with one or more display generation components. The one or more programs include instructions for: displaying, via the one or more display generation components, a respective user interface; and while displaying the respective user interface: in accordance with a determination that a set of respiratory disturbance criteria is satisfied over a period of time, displaying, via the one or more display generation components, a notification corresponding to one or more respiratory disturbances, wherein the set of respiratory disturbance criteria includes a set of sleep session criteria that is satisfied when at least a threshold amount of sleep sessions from a set of sleep sessions have at least a threshold amount of respiratory disturbances; and in accordance with a determination that the set of respiratory disturbance criteria is not satisfied for the period of time, forgoing display of the notification corresponding to the one or more respiratory disturbances.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for displaying respiratory disturbances, thereby increasing the effectiveness, efficiency, and user satisfaction with such devices. Such methods and interfaces may complement or replace other methods for displaying respiratory disturbances.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (APIs) to perform operations.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6V illustrate exemplary user interfaces for controlling display of respiratory disturbances, in accordance with some embodiments.

FIG. 7 is a flow diagram illustrating a method for controlling display of respiratory disturbances, in accordance with some embodiments.

FIG. 8 is a flowchart of a process for initiating a notification using a computer system, in accordance with some embodiments.

FIGS. 9A-9B illustrate graphs of sleep data for sleep sessions, in accordance with some embodiments.

FIG. 10 is a flow diagram illustrating a method for displaying a respiratory disturbance notification, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

Electronic devices are used to detect respiratory disturbances. There is a need for electronic devices that provide efficient methods and interfaces for displaying respiratory disturbances. Such techniques can reduce the cognitive burden on a user who accesses respiratory disturbances, thereby enhancing productivity. Further, such techniques can reduce processor and battery power otherwise wasted on redundant user inputs.

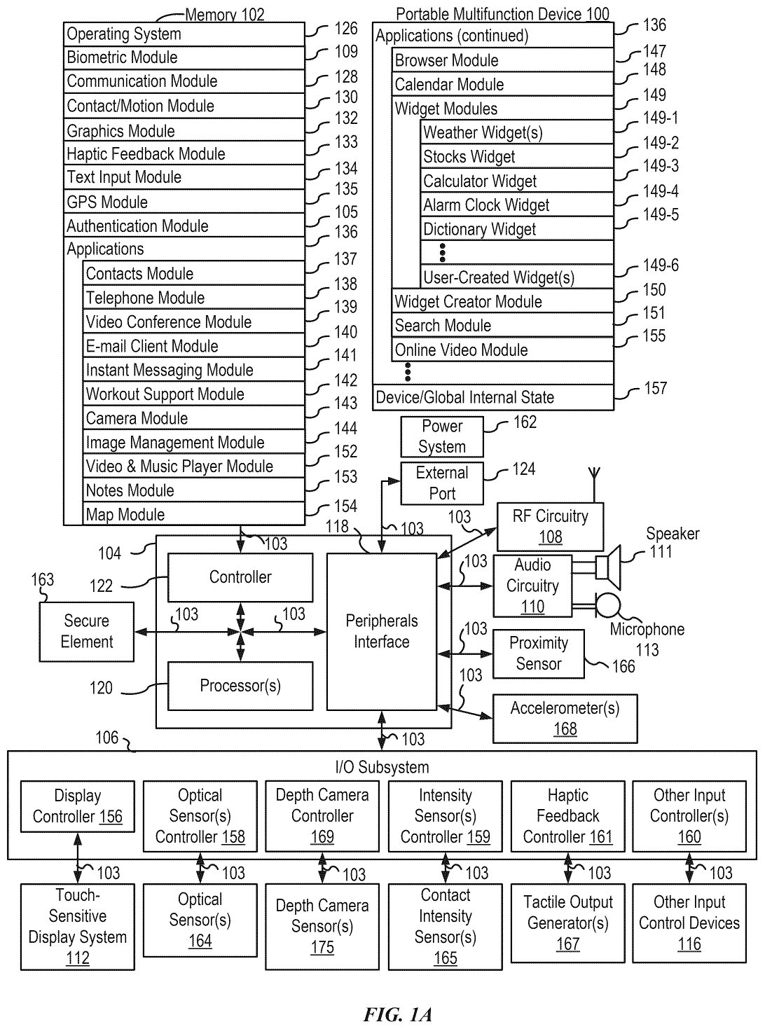

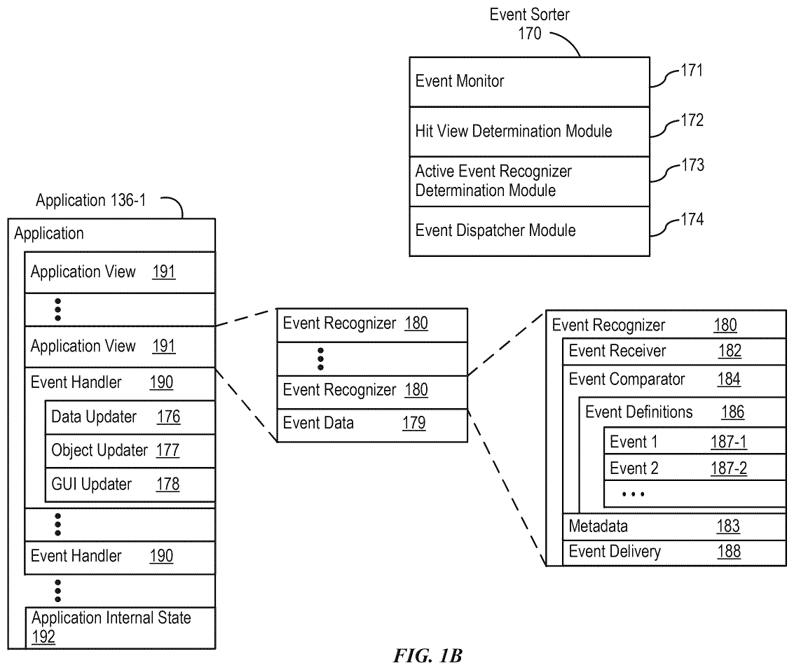

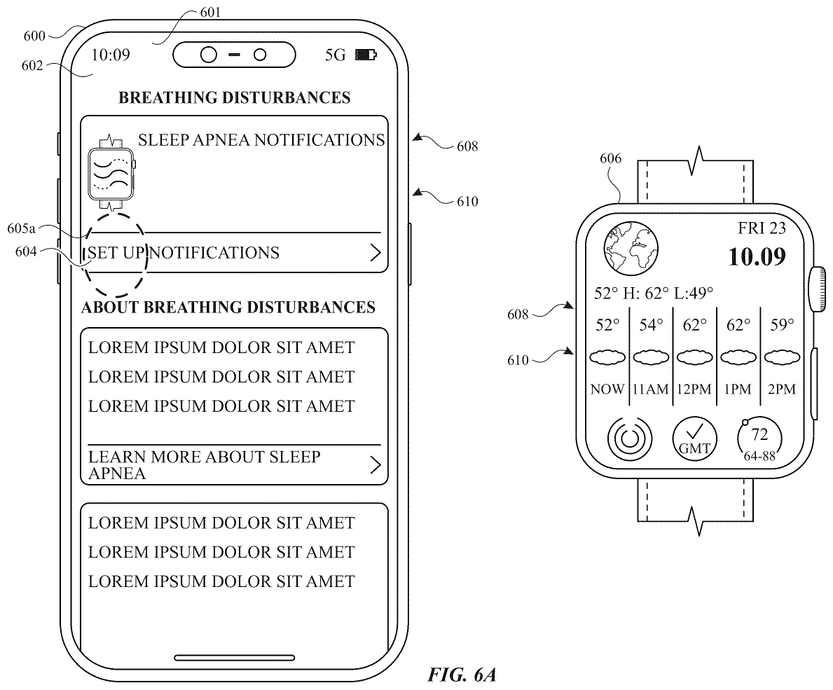

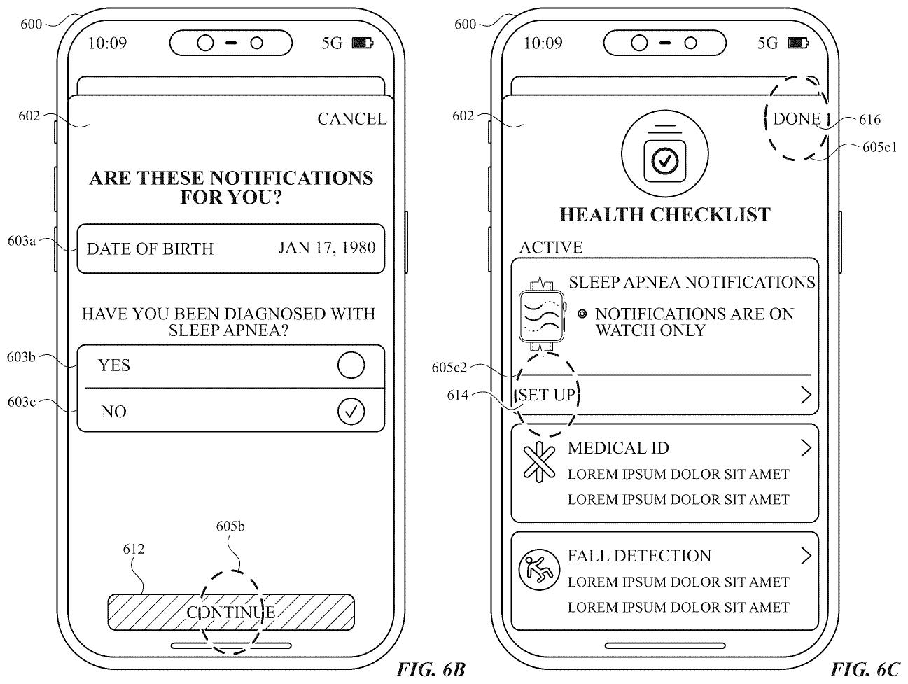

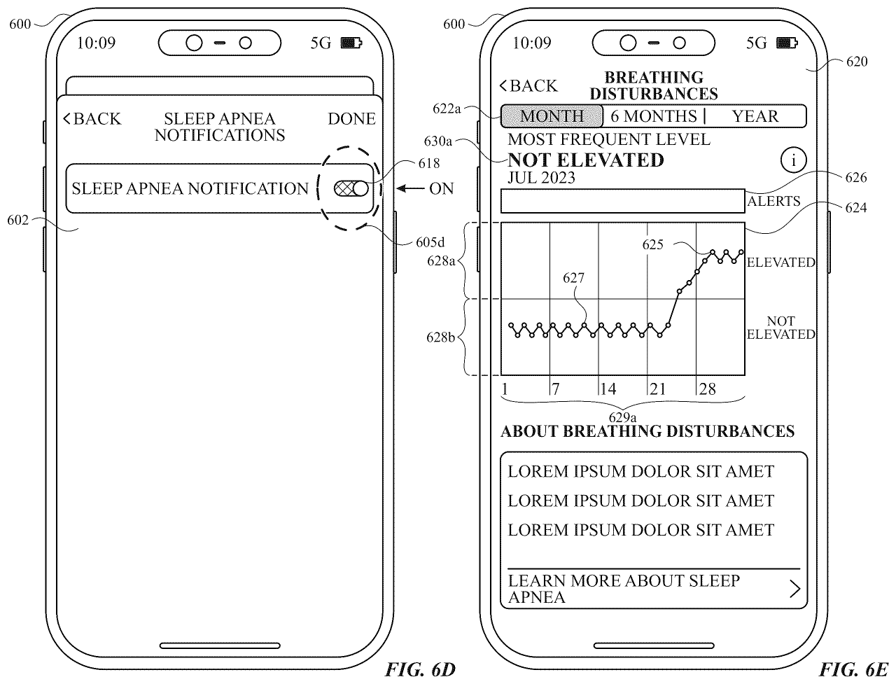

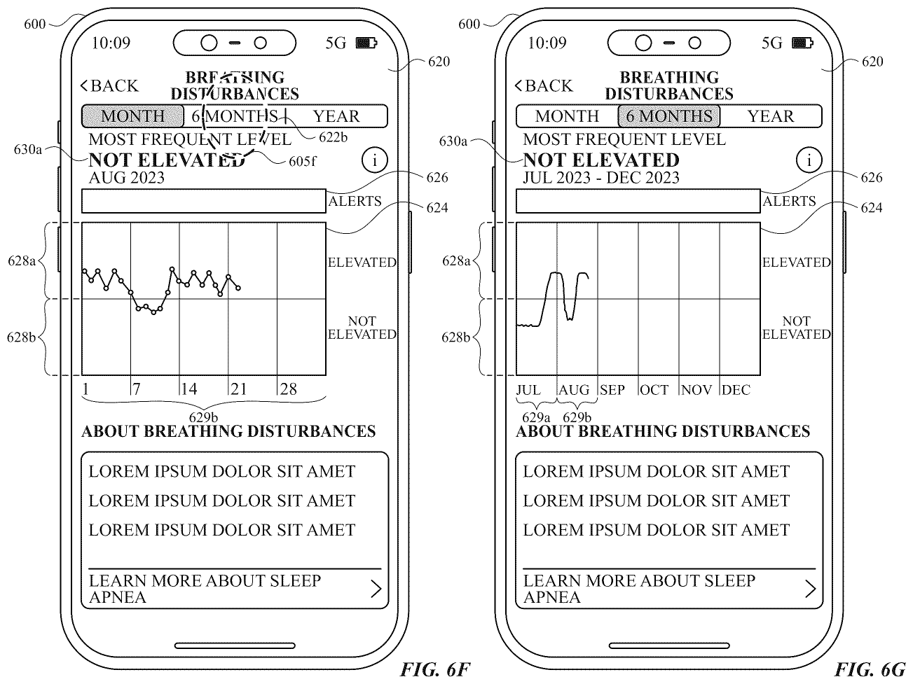

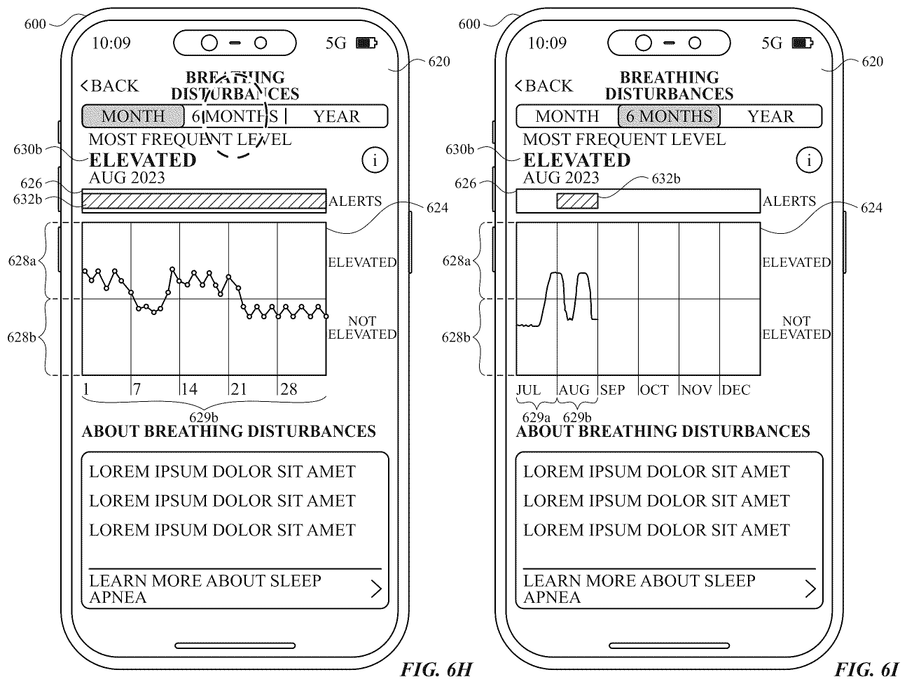

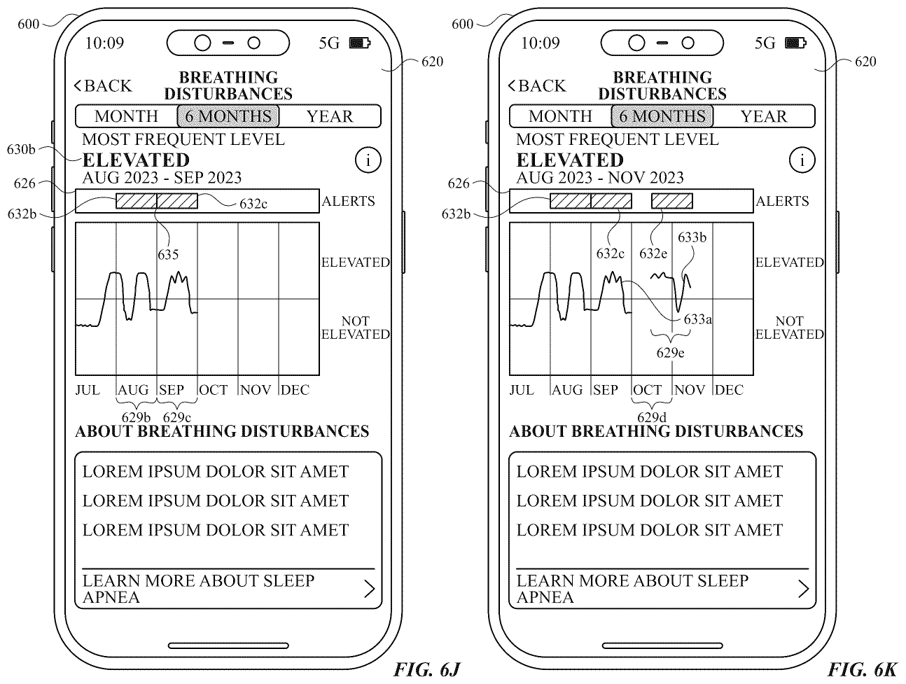

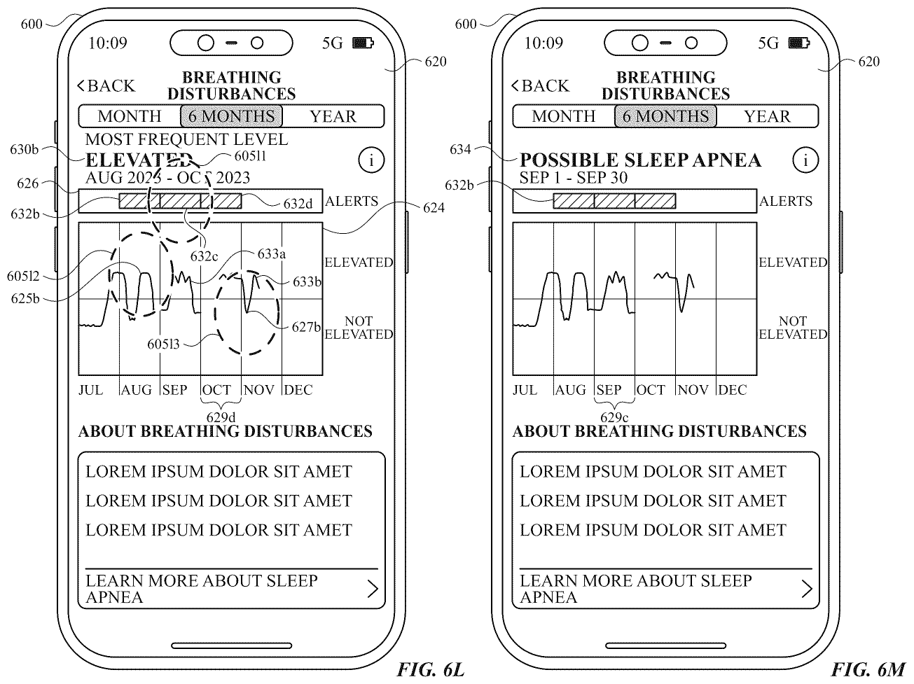

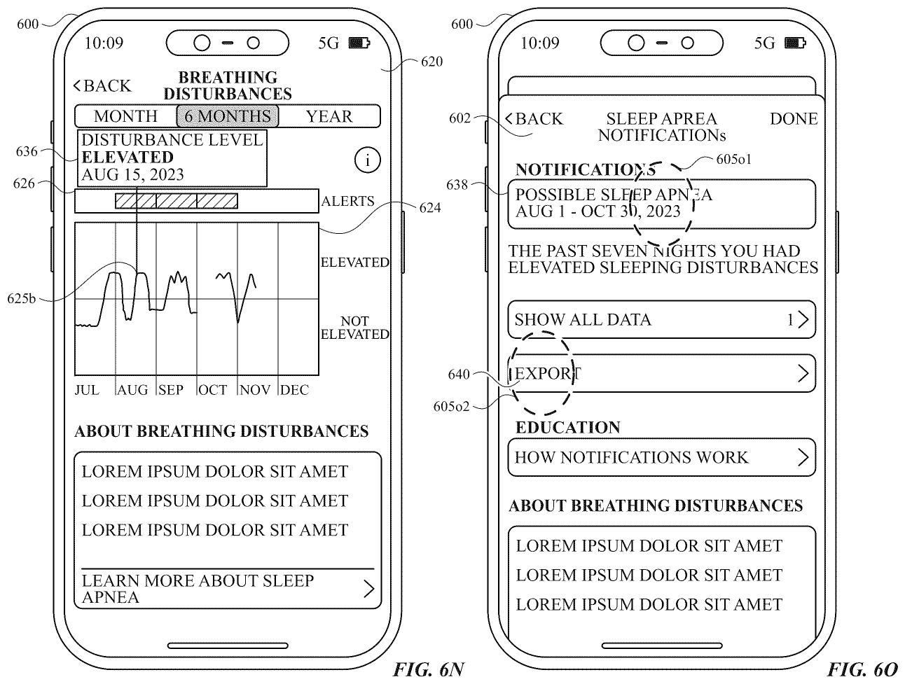

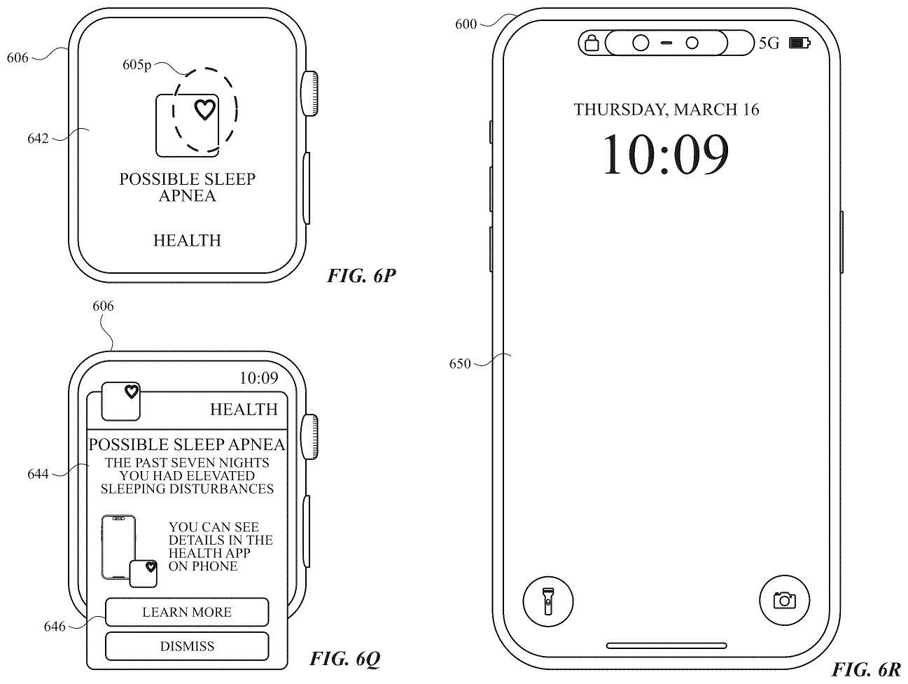

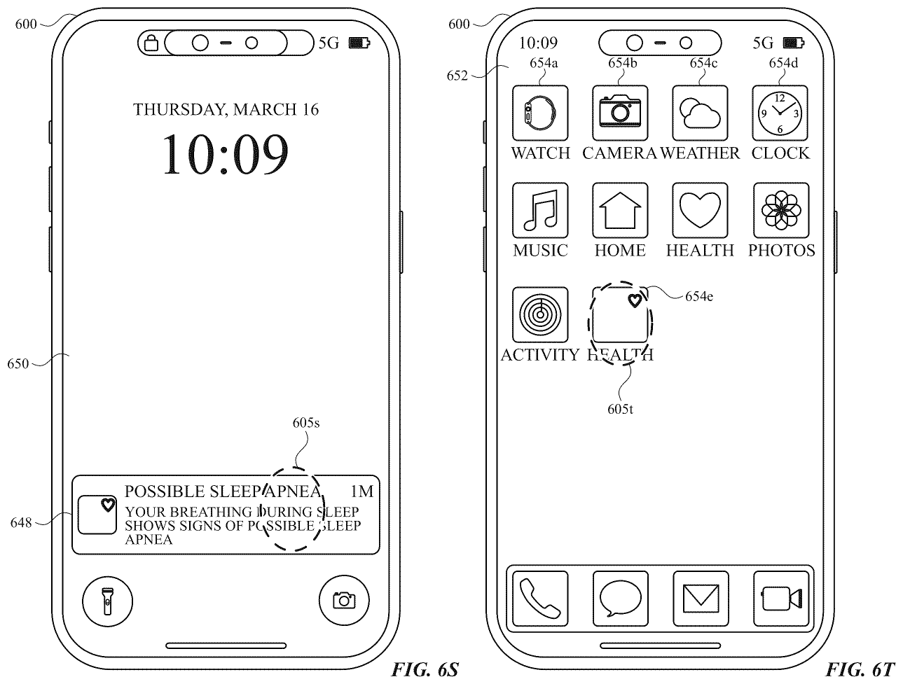

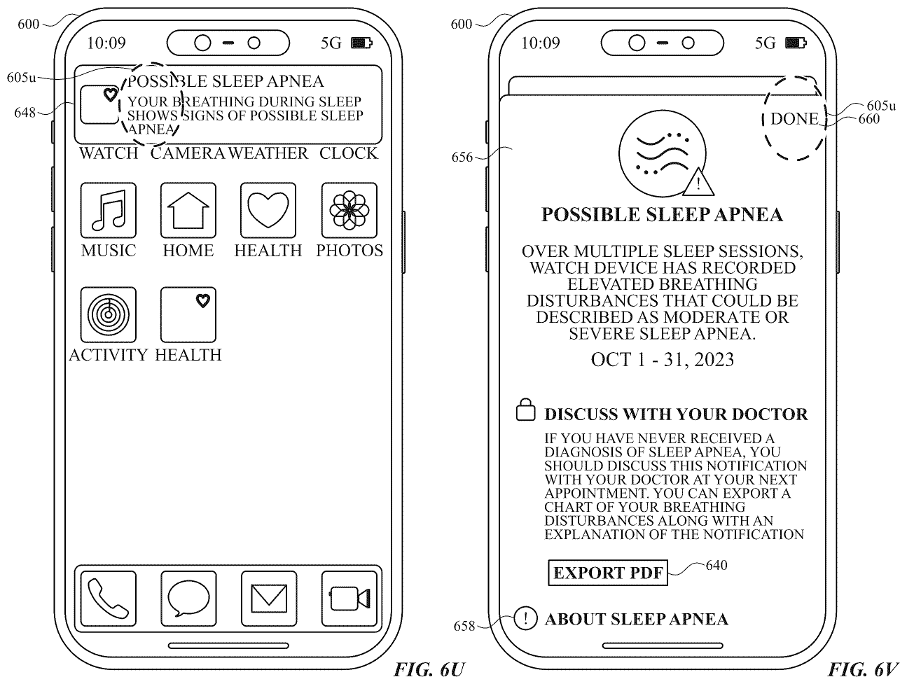

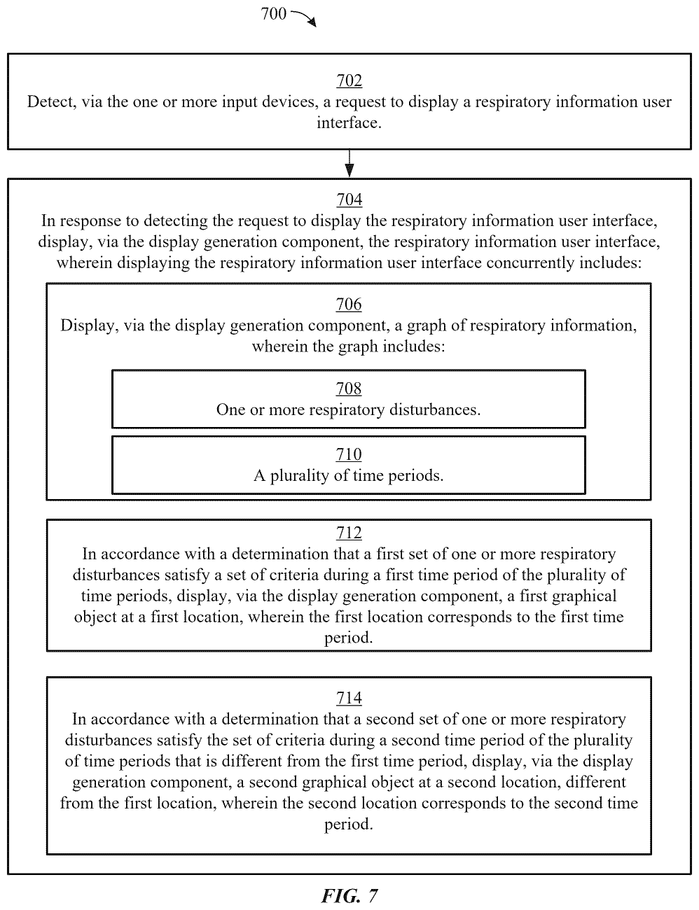

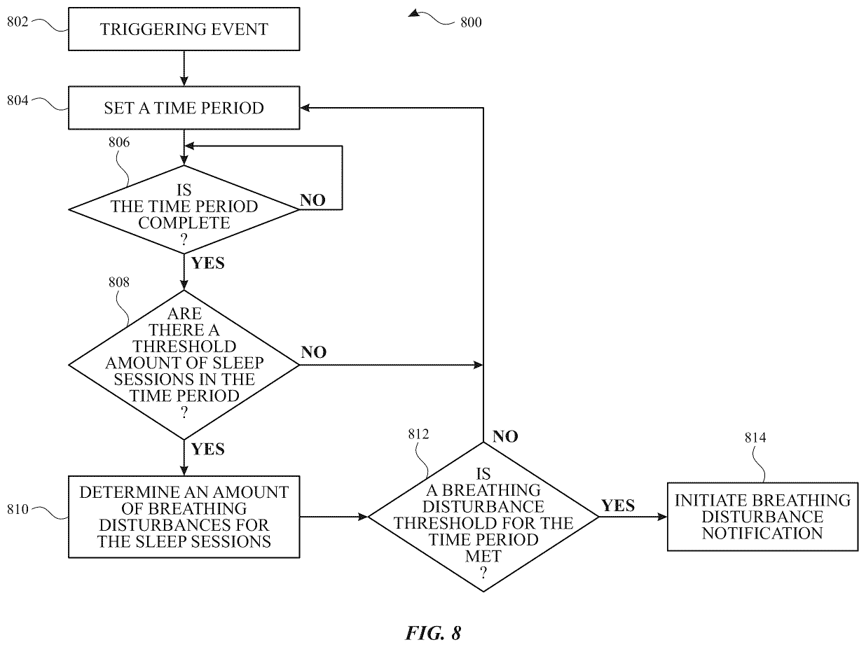

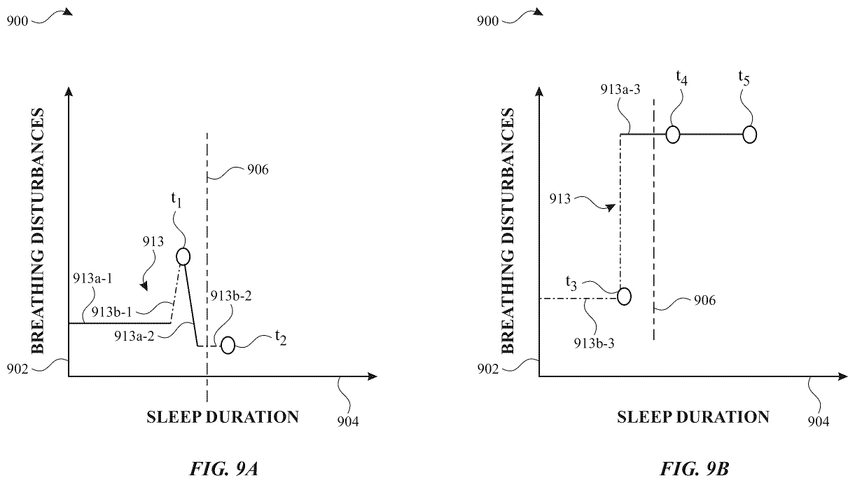

Below, FIGS. 1A-1B, 2, 3A-3G, 4A-4B, and 5A-5B provide a description of exemplary devices for performing the techniques for displaying respiratory disturbances. FIGS. 6A-6V illustrate exemplary user interfaces for controlling display of respiratory disturbances, in accordance with some embodiments. FIG. 7 is a flow diagram illustrating a method for controlling display of respiratory disturbances, in accordance with some embodiments. FIG. 8 is a flowchart of a process for initiating a notification using a computer system, in accordance with some embodiments. FIGS. 9A-9B illustrate graphs of sleep data for sleep sessions, in accordance with some embodiments. The user interfaces in FIGS. 6A-6V are used to illustrate the processes described below, including the processes in FIGS. 7, 8, and 10.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component (e.g., a display device such as a head-mounted display (HMD), a display, a projector, a touch-sensitive display, or other device or component that presents visual content to a user, for example on or in the display generation component itself or produced from the display generation component and visible elsewhere). The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

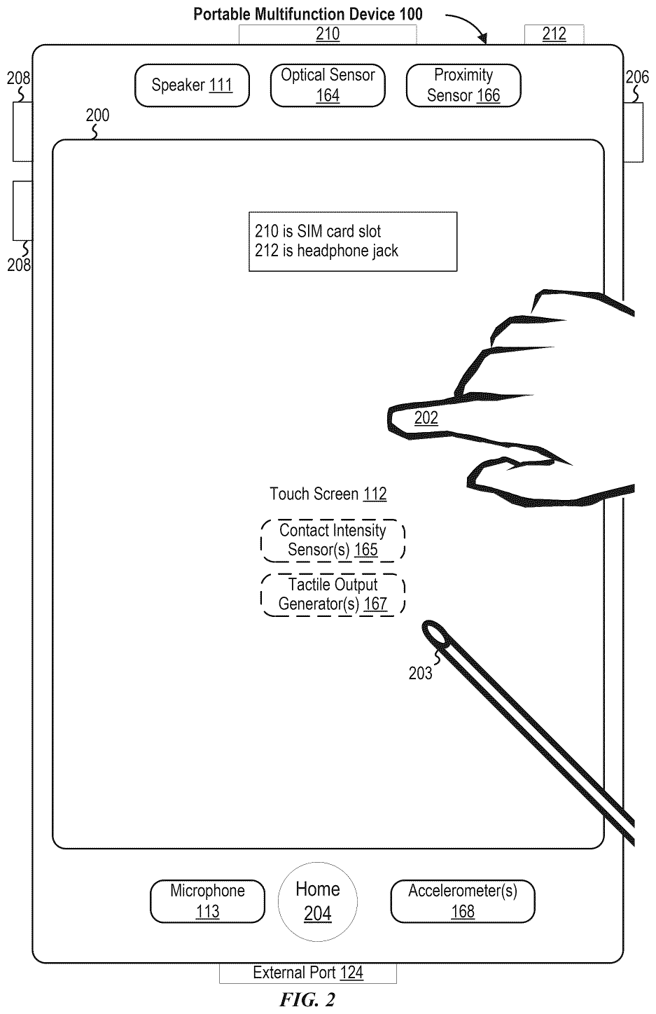

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both cars) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

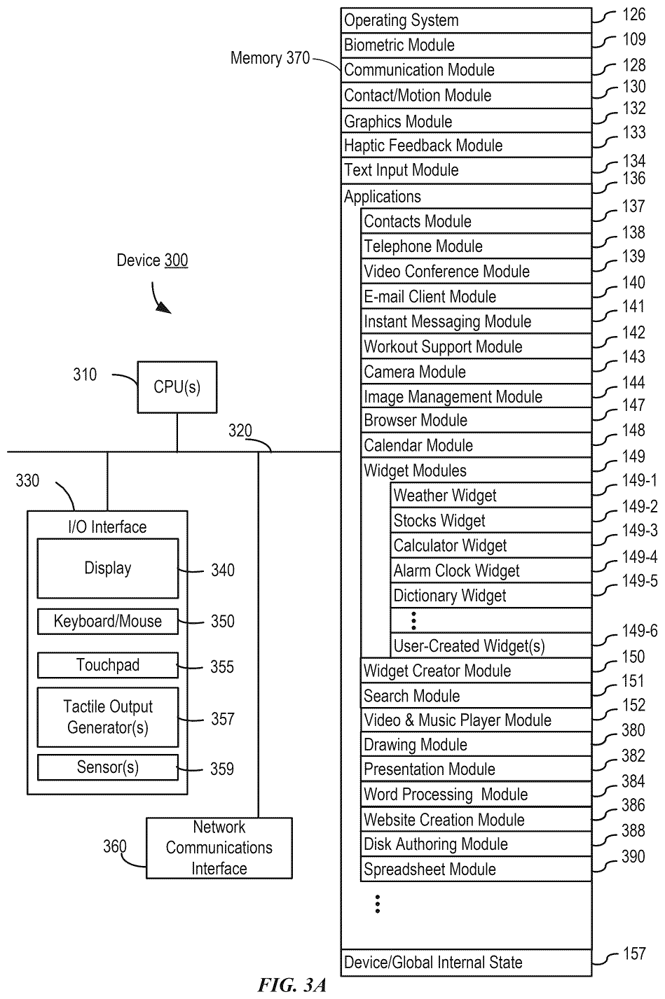

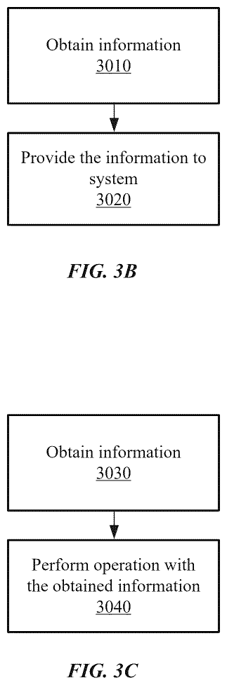

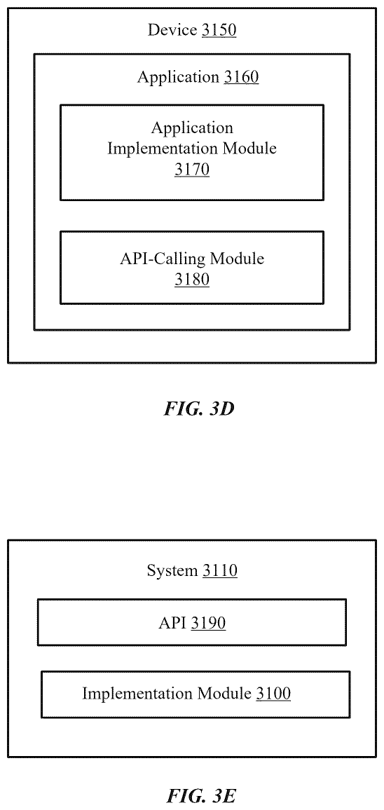

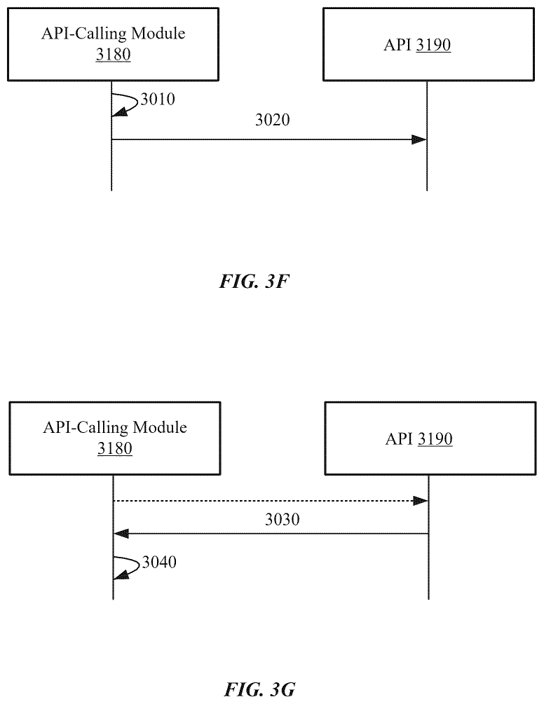

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136. Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3D, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HMD) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls APIs from different providers such as a set of APIs from an OS provider, another set of APIs from a plug-in provider, and/or another set of APIs from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IMU (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private APIs or partner APIs) are APIs that are accessible to a limited set of software processes (e.g., only software processes within an operating system or only software processes that are approved to access the limited APIs). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some APIs enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more APIs (e.g., via multiple different APIs). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

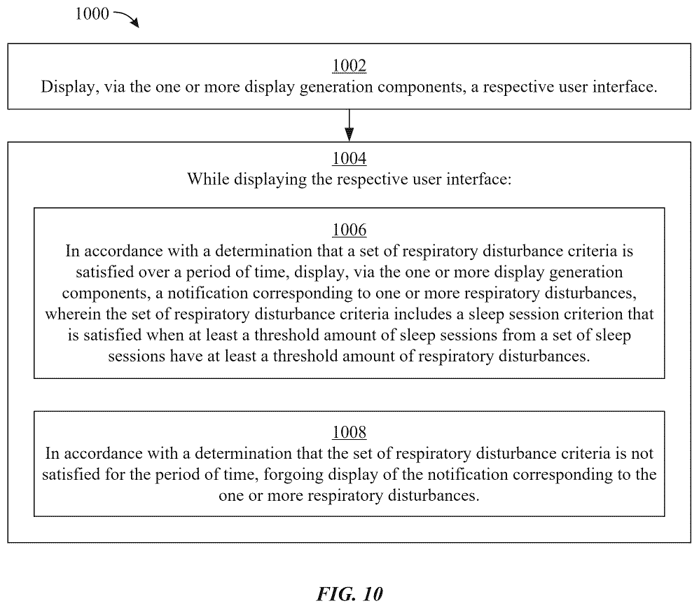

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform method 700 (e.g., FIG. 7), process 800 (e.g., FIG. 8), and/or method 1000 (e.g., FIG. 10) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module 3180) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

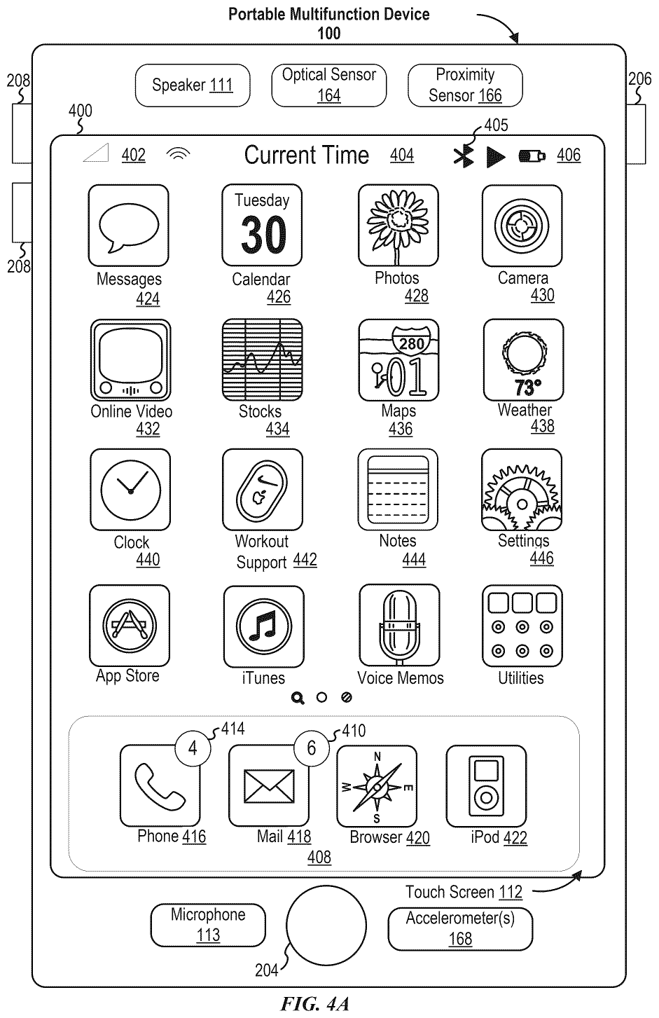

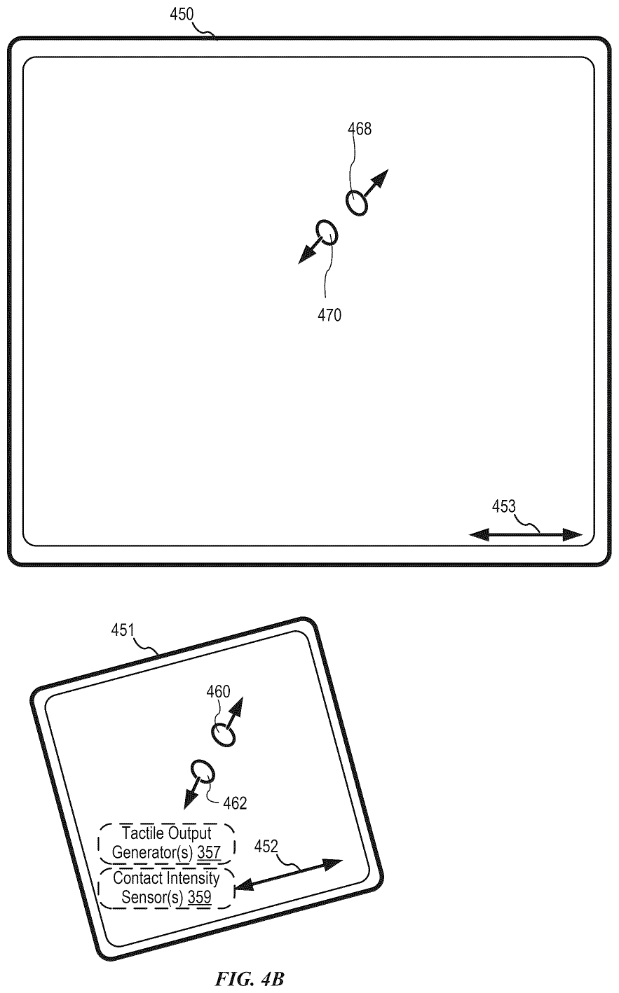

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

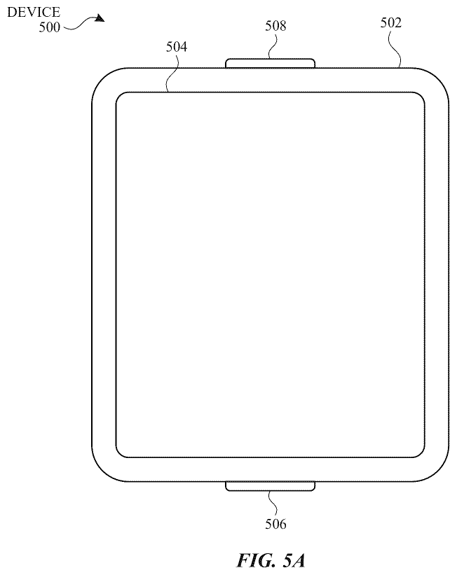

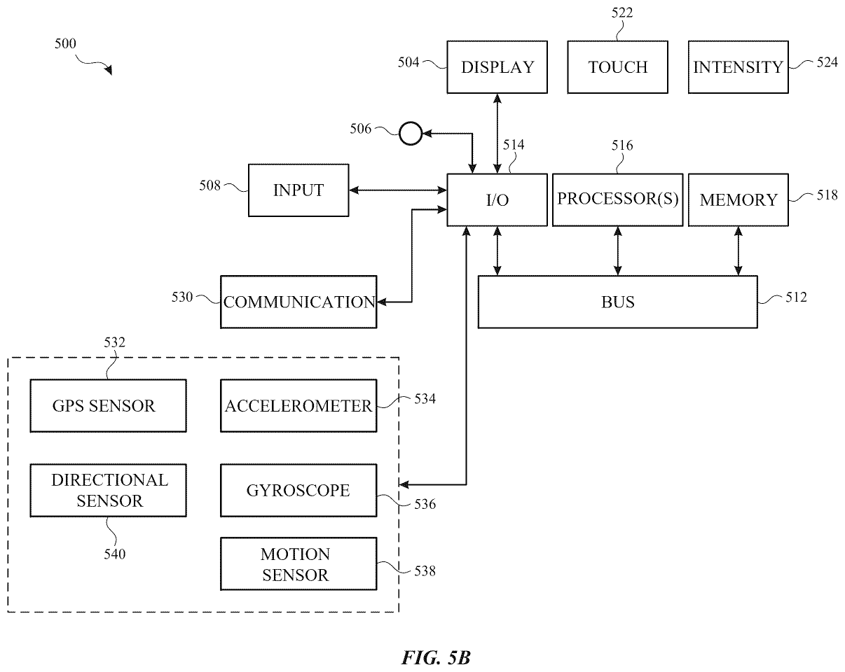

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including process 700 (FIG. 7). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

In some embodiments, the computer system is in a locked state or an unlocked state. In the locked state, the computer system is powered on and operational but is prevented from performing a predefined set of operations in response to user input. The predefined set of operations optionally includes navigation between user interfaces, activation or deactivation of a predefined set of functions, and activation or deactivation of certain applications. The locked state can be used to prevent unintentional or unauthorized use of some functionality of the computer system or activation or deactivation of some functions on the computer system. In some embodiments, in the unlocked state, the computer system is powered on and operational and is not prevented from performing at least a portion of the predefined set of operations that cannot be performed while in the locked state. When the computer system is in the locked state, the computer system is said to be locked. When the computer system is in the unlocked state, the computer is said to be unlocked. In some embodiments, the computer system is in the locked state optionally responds to a limited set of user inputs, including input that corresponds to an attempt to transition the computer system to the unlocked state or input that corresponds to powering the computer system off.

Attention is now directed towards embodiments of user interfaces (“UI”) and associated processes that are implemented on an electronic device, such as portable multifunction device 100, device 300, or device 500.

FIGS. 6A-6V illustrate exemplary user interfaces for controlling display of respiratory disturbances, in accordance with some embodiments. The user interfaces in these figures are used to illustrate the processes described below, including the processes in FIGS. 7, 8, and 10. In some embodiments, device 600 and wearable device 606 of FIGS. 6A-6V includes one or more features of portable multifunction device 100, device 300, or device 500. Although the description of FIGS. 6A-6V refers to device 600 and/or wearable device 606 making certain determinations, those determinations can alternatively or additionally be made by other devices, such as by a remote device and/or a server.

At FIG. 6A, device 600 displays, on display 601, interface 602. Interface 602 includes set up notifications option 604. Set up notifications option 604, when activated, initiates a process to enable device 600 to display information corresponding to breathing disturbances. As described herein, device 600 displays various graphical elements for breathing disturbances. In some embodiments, the breathing disturbance includes a hypopnea and/or apnea. In some embodiments, the breathing disturbance includes a set of one or more shallow breaths and/or a set of one or more stoppages of breathing. In some embodiments, the breathing disturbance occurs while a person is sleeping. In some embodiments, the breathing disturbance is detected via one or more sensors of device 600 and/or wearable device 606 while a person is wearing the device. In some embodiments, the breathing disturbance is detected via accelerometer 608 and/or blood oxygen sensor 610 (e.g., one or more cameras that detect blood oxygen and/or an oximeter). In some embodiments, the breathing disturbance is detected via accelerometer 608 and is not detected via blood oxygen sensor 610 (and/or is not detected via a process to test blood oxygen level). In some embodiments, device 600 and/or wearable device 606 determines if a set of breathing disturbance criteria (e.g., rules and/or patterns) is satisfied based on movement that is detected via accelerometer 608 that corresponds to a person's breathing pattern. In some embodiments, device 600 receives (e.g., via a communications network) the information for detecting a breathing disturbance from wearable device 606. At FIG. 6A, device 600 detects input 605a (e.g., a touch input, an air gesture, a button press, a gaze, and/or a speech input) directed at set up notifications option 604.

At FIG. 6B, in response to detecting input 605a, device 600 displays interface 602. Interface 602 at FIG. 6B includes option 603a that is configured to receive a date of birth and options 603b and 603c that are configured to receive information corresponding to whether a person has been diagnosed with sleep apnea. At FIG. 6B, device 600 detects input 605b (e.g., a touch input, an air gesture, a button press, a gaze, and/or a speech input) directed at continue option 612. In response to detecting input 605b, device 600 displays interface 602 of FIG. 6C. In some embodiments, if device 600 determines that a person has been diagnosed with sleep apnea, breathing disturbance information described herein is not displayed.

At FIG. 6C, interface 602 indicates that notifications are turned on. In some embodiments, device 600 detects input 605cl (e.g., a touch input, an air gesture, a button press, a gaze, and/or a speech input) directed at done option 616 and, in response, begins monitoring for breathing disturbance information. At FIG. 6C, device 600 detects input 605c2 (e.g., a touch input, an air gesture, a button press, a gaze, and/or a speech input) directed at notification setting option 614. In response to detecting input 605c2, device 600 displays interface 602 at FIG. 6D.

At FIG. 6D, interface 602 is configured to enable and/or disable breathing disturbance notifications. For example, breathing disturbance notifications are enabled and/or disabled in response to device 600 detecting input 605d (e.g., a touch input, an air gesture, a button press, a gaze, and/or a speech input) directed at notification setting 618.

At FIG. 6E, device 600 displays interface 620, which includes breathing disturbance information. In some embodiments, device 600 displays interface 620 in response to detecting input 605cl. Interface 620 includes a month-view of graph 624 and alert area 626 (as depicted by month view option 622a being selected). Alert area 626 is displayed above graph 624. Graph 624 includes elevated region 628a (e.g., along the positive y-axis) and non-elevated region 628b (e.g., along the negative y-axis). Graph 624 also includes a timeline along the x-axis. At FIG. 6E, the timeline includes time window 629a. As described herein, device 600 determines whether the person associated with device 600 has had elevated breathing disturbances a threshold amount of times within a particular time window (e.g., time window 629a). Time window 629a begins on July 1^{st} and ends on July 31^{st} based on breathing disturbance information being available starting July 1^{st} through July 31^{st}. In some embodiments, time window 629a begins on a day at which device 600 is configured to begin detecting breathing disturbance information. As described in greater detail herein, in some embodiments, breathing disturbance information is not available and, as such, device 600 shifts the time window. In some embodiments, time window 629a begins and ends on a day that is different from July 1^{st} and July 31^{st}. In some embodiments, time window 629a is a window of time and/or a time period (e.g., an uninterrupted time period) that is based on a range of days (e.g., 30 days) having a threshold amount of breathing disturbance information, as described in greater detail herein.

At FIG. 6E, graph 624 includes non-elevated graphical object 627 and elevated graphical object 625. Non-elevated graphical object 627 and elevated graphical object 625 illustrate whether breathing disturbance criteria has been satisfied (e.g., for a particular day, night, and/or sleep session in time window 629a). For example, non-elevated graphical object 627 indicates that breathing disturbance criteria has not been satisfied. Elevated graphical object 625 indicates that breathing disturbance criteria has been satisfied. In some embodiments, non-elevated graphical object 627 and/or elevated graphical object 625 are discrete objects (e.g., shapes and/or icons). In some embodiments, non-elevated graphical object 627 and/or elevated graphical object 625 are discrete objects displayed along a continuous portion of a line.

At FIG. 6E, device 600 does not display an alert indicator (such as alert indicator 632b in FIGS. 6H-6I) in alert area 626. In some embodiments, device 600 does not display a graphical object in alert area 626 because a threshold amount of breathing disturbances (e.g., 5, 10, 15, and/or 20) have not occurred during time window 629a. For example, graph 624 includes multiple elevated graphical objects 625, indicating that multiple breathing disturbances (e.g., breathing disturbance events) have been detected. Even though multiple breathing disturbances have been detected, device 600 does not display a graphical object in alert area 626 because the threshold amount of breathing disturbances has not been met for time window 629a. Moreover, device 600 displays non-elevated indicator 630a that indicates the threshold amount of breathing disturbances has not been met for time window 629a.

At FIG. 6E, to determine whether elevated graphical object 625 should be displayed, device 600 determines if the person associated with device 600 has had an elevated breathing disturbance level for multiple intervals of time during a sleep session (e.g., the person has had an elevated breathing disturbance for multiple one-hour intervals). In some embodiments, device 600 determines whether a threshold amount (e.g., 3, 5, and/or 9) of breathing disturbances has occurred during a particular interval of time (e.g., 30 minutes, 1 hour, and/or 2 hours) and/or on a per interval basis. In some embodiments, device 600 determines whether the threshold amount (e.g., 3, 5, and/or 9) of breathing disturbances is satisfied for each interval of time during a sleeping session. In some embodiments, device 600 determines a sleeping session occurs when device 600 determines a person is still for a threshold amount of time (e.g., 30 minutes and/or 1 hour).

At FIG. 6E, in some embodiments, after determining the threshold amount of breathing disturbances has occurred on a per interval basis (e.g., per sampling window basis), device 600 determines whether the number of intervals that have elevated breathing disturbances satisfy a threshold amount of intervals of time (e.g., a threshold amount of 1 interval, 3 intervals, or 4 intervals). If the number of intervals that have elevated breathing disturbances meet or exceed the threshold amount of intervals of time, device 600 determines that an elevated breathing disturbance has occurred and, as such, displays elevated graphical object 625. If the number of intervals that have elevated breathing disturbances do not meet or exceed the threshold amount of intervals of time, device 600 determines that an elevated breathing disturbance has occurred and, as such, displays non-elevated graphical object 627.

At FIG. 6E, in some embodiments, the threshold amount of intervals of time is based on a mode (e.g., a statistical mode) of elevated breathing disturbances (e.g., and is not based on an average of elevated breathing disturbances). In some embodiments, the threshold amount of intervals of time needed to display elevated graphical object 625 is based on a ratio (e.g., comparison) of elevated breathing disturbances relative to non-elevated breathing disturbances. For example, in some embodiments, device 600 determines an amount of intervals that have elevated breathing disturbances (e.g., the person has had five one-hour intervals where each one-hour interval includes more than 5 hypopneas) compared to an amount of intervals that have non-elevated breathing disturbances (e.g., the person has had three one-hour intervals where each one-hour interval includes less than 5 hypopneas). In some embodiments, based on the comparison of the amount of intervals that have elevated breathing disturbances and the amount of intervals that have non-elevated breathing disturbances, device 600 determines whether an elevated breathing disturbance has occurred.

At FIG. 6F, device 600 has received additional breathing disturbance information. As depicted, graph 624 includes breathing disturbance information for time window 629b. Device 600 determines breathing disturbance information for time window 629b meets and/or exceeds a threshold amount of breathing disturbances (e.g., 10, 12, and/or 15) for time window 629b. However, device 600 does not display a graphical object in alert area 626 until time window 629b is complete (e.g., until the last day of time window 629b has passed). In some embodiments, device 600 provides a notification (e.g., a weekly notification and/or a daily notification), such as notification 642 and/or notification 648 of FIGS. 6P-6T, that elevated breathing disturbances have been detected in time window 629b while not displaying a graphical object in alert area 626 in graph 624. At FIG. 6F, device 600 detects input 605f (e.g., a button press, an air gesture, a gaze, and/or a touch gesture) directed at six-month view option 622b. In response to detecting input 605f, device 600 displays interface 620 of FIG. 6G.

At FIG. 6G, interface 620 is a six-month view of breathing disturbance information. Graph 624 includes breathing disturbance information for time window 629a that is described with respect to FIG. 6E and breathing disturbance information for time window 629b that is described with respect to FIG. 6F. As depicted, device 600 does not display a graphical object in alert area 626 corresponding to time window 629a and/or time window 629b. While not illustrated, in some embodiments, graph 624 of FIG. 6G includes one or more non-elevated graphical objects (e.g., similar to non-elevated graphical object 627 of FIG. 6E) and/or one or more elevated graphical objects (e.g., similar to elevated graphical object 625 of FIG. 6E).

At FIGS. 6H-61, device 600 determines that time window 629b is complete. Device 600 has also received additional breathing disturbance information for time window 629b. At FIGS. 6H-6I, device 600 determines that the breathing disturbance information for time window 629b satisfies a threshold level of breathing disturbances (also referred to as “threshold breathing disturbance level”). In some embodiments, device 600 determines that the breathing disturbance information for time window 629b satisfies the threshold breathing disturbance level based on determining that the threshold amount of intervals is satisfied for a set of sleep sessions (e.g., during a day and/or night) throughout time window 629b (e.g., the person has had breathing disturbances for at least 5, 10, and/or 15 sleep sessions during a thirty-day window). In response to detecting that the breathing disturbance information for time window 629b satisfies the threshold breathing disturbance level, device 600 displays alert indicator 632b in alert area 626.

At FIGS. 6H-6I, device 600 displays alert indicator 632b based on a position of time window 629b in graph 624. At FIGS. 6H-6I, time window 629b starts on August 1^{st} and ends on August 31^{st}. As depicted in FIGS. 6H-6I, August 1^{st} and August 31^{st} have a respective position on graph 624. Device 600 displays alert indicator 632b at a position that is based on the respective position of August 1^{st} and August 31^{st} on graph 624. As depicted, alert indicator 632b is displayed at a position that is directly above the respective position of August 1^{st} and August 31^{st} on graph 624. In some embodiments, time window 629b starts and ends on calendar days that are different from August 1^{st} and August 31^{st}. In some embodiments, device 600 displays alert indicator 632b at a position that is above a respective position of the days in which time window 629b starts and ends. At FIG. 6I, for the six-month view of interface 620, device 600 does not display a portion of alert indicator 632b at an area of interface 620 that overlaps with an area directly above time window 629a.

At FIGS. 6H-6I, the length of alert indicator 632b is based on (e.g., is dependent on and/or determined from) the length of time window 629b. As depicted, the length of alert indicator 632b extends throughout the entire length of time window 629b. In some embodiments, the length of alert indicator 632b is at least 70%, 80%, 90%, and/or 95% the length of time window 629b.

At FIGS. 6H-6I, device 600 displays elevated indicator 630b in response to detecting that the breathing disturbance information for time window 629b satisfies the threshold breathing disturbance level. As depicted, device 600 displays elevated indicator 630b without displaying non-elevated indicator 630a.

At FIG. 6J, breathing disturbance information is received for time window 629c. At FIG. 6J, device 600 determines that the threshold breathing disturbance level is satisfied for time window 629c. In response to determining that the threshold breathing disturbance level is satisfied for time window 629c, device 600 displays alert indicator 632c. Alert indicator 632c is displayed based on a position of time window 629c and/or has a length that is based on the length of time window 629c. In some embodiments, alert indicator 632c and alert indicator 632b do not overlap (e.g., and/or are adjacent to one another). Alert indicator 632c and alert indicator 632b are visually distinguished from each other. In some embodiments, alert indicator 632c and alert indicator 632b are visually distinguished based on having visually distinguishable shapes. In some embodiments, alert indicator 632c and alert indicator 632b are visually distinguished based on having a boundary (e.g., boundary 635) (or, optionally, a gap) between alert indicator 632c and alert indicator 632b.

At FIG. 6J, device 600 does not display a portion of alert indicator 632b at an area of interface 620 that overlaps with an area above time window 629c. Device 600 does not display a portion of alert indicator 632c at an area of interface 620 that overlaps with an area above (e.g., directly above) time window 629b.

At FIG. 6K, device 600 shifts a time window to account for the availability of breathing disturbance information. In some embodiments, breathing disturbance information is not available because wearable device 606 was not worn by a person while sleeping. As depicted by the gap between breathing disturbance information 633a and breathing disturbance information 633b, breathing disturbance information is not available at the beginning of October. In some embodiments, device 600 determines a threshold amount (e.g., 2 days, 5 days, and/or 15 days) of breathing disturbance information is not available and, in response, shifts the time window. For example, in some embodiments, time window 629e starts at the beginning of breathing disturbance information 633b (e.g., time window 629e starts in the middle of October as opposed to starting at the beginning of October, as depicted by time window 629d). In some embodiments, device 600 determines if breathing disturbance information 633b satisfies the threshold breathing disturbance level, as described in greater detail with respect to FIGS. 6H-61. At FIG. 6K, device 600 determines that the threshold breathing disturbance level is satisfied and, in response, displays alert indicator 632c. In some embodiments, device 600 displays alert indicator 632e at a position that corresponds to time window 629e (e.g., alert indicator 632c starts in the middle of October as opposed to starting at the beginning of October, as depicted by alert indicator 632d of FIG. 6L). As depicted, device 600 displays alert indicator 632e in an area above (e.g., directly above) time window 629e. As depicted, alert indicator 632e starts at the beginning of breathing disturbance information 633b, similar to time window 629e. Moreover, alert indicator 632e is spaced apart from alert indicator 632c. In some embodiments, device 600 receives additional breathing disturbance information beyond time window 629c. In such embodiments, device 600 determines a new time window based on the shifting of time window 629e, where the new time window occurs at the end of time window 629e (e.g., so long as a threshold amount of breathing disturbance information is available to determine whether the threshold breathing disturbance level is satisfied).

At FIG. 6L, device 600 does not shift a time window to account for breathing disturbance information not being available. For example, in some embodiments, time window 629d starts at the beginning of October as opposed to starting at the start of breathing disturbance information 633b (e.g., time window 629d does not start in the middle of October). In some embodiments, device 600 determines that breathing disturbance information 633b satisfies the threshold breathing disturbance level even though a portion of time window 629d does not include breathing disturbance information.

At FIG. 6L, device 600 determines that the threshold breathing disturbance level is satisfied and, in response, displays alert indicator 632d. In some embodiments, device 600 displays alert indicator 632d at a position that corresponds to time window 629d (e.g., alert indicator 632d starts at the beginning of October as opposed to starting in the middle of October, as depicted by alert indicator 632e of FIG. 6K). As depicted, device 600 displays alert indicator 632d in an area above (e.g., directly above) time window 629d. Moreover, alert indicator 632d is not spaced apart from alert indicator 632c.

At FIG. 6L, alert indicator 632d is a continuous (e.g., uninterrupted) graphical object regardless of a portion of the breathing disturbance information not being available. In some embodiments, device 600 displays alert indicator 632d as a continuous object when device 600 determines that a threshold amount of breathing disturbance information is available (e.g., even though some breathing disturbance information is not available for one or more sleep sessions and/or calendar days, there is enough breathing disturbance information for device 600 to determine whether the threshold breathing disturbance level is satisfied for time window 629d).

At FIG. 6L, alert indicators 632b, 632c, and 632d are selectable objects. For example, in some embodiments, selecting an alert indicator causes device 600 to display a different graphical user interface (e.g., update a currently displayed interface and/or replace a currently displayed interface with a different interface). Device 600 detects input 60511 (e.g., a touch input, an air gesture, a button press, a gaze, and/or a speech input) directed toward alert indicator 632c. In response to detecting input 60511, device 600 displays interface 620 of FIG. 6M.

At FIG. 6M, in response to detecting input 60511, device 600 displays possible sleep apnea indicator 634 without displaying elevated indicator 630b. In some embodiments, possible sleep apnea indicator 634 indicates a potential and/or risk of a medical condition (e.g., sleep apnea). Moreover, in response to detecting input 60511, device 600 displays a representation of time window 629c (e.g., “September 1-September 30”) (and, optionally, without displaying a representation of a period of time of an elevated level of breathing disturbance, such as “August 2023-October 2023” depicted in FIG. 6L).

Returning to FIG. 6L, breathing disturbance information 633a and 633b are selectable objects. For example, in some embodiments, selecting breathing disturbance information 633a and/or breathing disturbance information 633b causes device 600 to display a different graphical user interface (e.g., update a currently displayed interface and/or replace a currently displayed interface with a different interface). Device 600 detects input 60512 (e.g., a touch input, an air gesture, a button press, a gaze, and/or a speech input) directed toward elevated graphical object 625b of breathing disturbance information 633a. In response to detecting input 60512, device 600 displays interface 620 of FIG. 6N. In some embodiments, device 600 detects input 60513 (e.g., a touch input, an air gesture, a button press, a gaze, and/or a speech input) directed toward non-elevated graphical object 627b of breathing disturbance information 633b. In response to detecting input 60513, device 600 displays an interface similar to interface 620 of FIG. 6N but includes “Not elevated, Nov. 1, 2023” without including elevated representation 636 of FIG. 6N (e.g., “Elevated, Aug. 15, 2023”).

At FIG. 6N, device 600 displays interface 620 with elevated representation 636. Elevated representation 636 includes a level of breathing disturbance (e.g., “elevated”) and a date for the breathing disturbance level (e.g., “Aug. 15, 2023”).

At FIG. 6O, device 600 displays interface 602, which is similar to interface 602 of FIG. 6A but has a different state. At FIG. 6O, interface 602 includes notification 638. Notification 638 indicates an issue corresponding to breathing disturbance level (e.g., “possible sleep apnea”). In some embodiments, device 600 displays notification 638 if device 600 determines that the threshold breathing disturbance level is satisfied for a time window. In some embodiments, device 600 displays notification 638 on a monthly basis (e.g., the threshold breathing disturbance level is satisfied for a particular month). In some embodiments, device 600 displays notification 638 on a weekly basis (e.g., a threshold breathing disturbance level is satisfied for a particular week). In some embodiments, device 600 detects input 60501 (e.g., a touch input, an air gesture, a button press, a gaze, and/or a speech input) directed at notification 638. In such embodiments, in response to detecting input 60501, device 600 displays an interface including a graph of breathing disturbance information and an alert area similar to interface 620 of FIGS. 6E-6L.

At FIG. 6O, interface 602 includes export option 640. In some embodiments, device 600 detects input 60502 (e.g., a touch input, an air gesture, a button press, a gaze, and/or a speech input) directed toward export option 640. In such embodiments, in response to detecting input 60502, device 600 initiates a process to export breathing disturbance information (e.g., breathing disturbance information 633a and/or breathing disturbance information 633b). In some embodiments, initiating a process to export breathing disturbance information includes exporting the breathing disturbance information to a PDF (e.g., portable document format). In some embodiments, initiating a process to export breathing disturbance information includes initiating a process to print the breathing disturbance information. In some embodiments, initiating a process to export breathing disturbance information includes exporting breathing disturbance information using an Apnea Hypopnea Index (AHI) (e.g., a number of apnea and/or hypopnea events per hour of sleep). In some embodiments, initiating a process to export breathing disturbance information includes initiating a communication to transmit breathing disturbance information to a computer system that is different from device 600 (e.g., a computer system associated with a health care provider).

At FIG. 6P, wearable device 606 displays notification 642. In some embodiments, wearable device 606 displays notification 642 in response to detecting the threshold breathing disturbance level is satisfied for a period of time (e.g., seven days and/or one month). In some embodiments, wearable device 606 displays notification 642 in response to detecting the threshold breathing disturbance level is satisfied for seven days. Notification 642 indicates an issue corresponding to breathing disturbance level (e.g., “possible sleep apnea”). Wearable device 606 detects input 605p (e.g., a touch input, an air gesture, a button press, a gaze, and/or a speech input) directed toward notification 642. In response to detecting 605p, wearable device 606 displays interface 644 of FIG. 6Q. In some embodiments, in response to detecting input 605p, wearable device 606 displays interface 620 of FIGS. 6E-6N, interface 602 of FIG. 6O, and/or interface 656 of FIG. 6V.

At FIG. 6Q, interface 644 includes information for an issue corresponding to breathing disturbance level (e.g., “possible sleep apnea”). Interface 644 includes information for a period of time in which a threshold breathing disturbance level is satisfied (e.g., “the past seven nights you had elevated sleeping disturbances”). Interface 644 includes learn more option 646. In some embodiments, in response to detecting an input (e.g., a touch input, an air gesture, a button press, a gaze, and/or a speech input) directed at learn more option 646, wearable device 606 displays a user interface corresponding to breathing disturbance information (e.g., how to view breathing disturbance information on device 600; and/or an interface similar to interface 620 of FIGS. 6E-6N, interface 602 of FIG. 6O, and/or interface 656 of FIG. 6V).

At FIG. 6R, device 600 displays locked user interface 650 while device 600 is in a locked state. In some embodiments, while in a locked state, device 600 is powered on and operational but is prevented from performing a predefined set of operations in response to user input. As depicted, locked user interface 650 does not include a notification, such as notification 648 of FIG. 6S. At FIG. 6R, in some embodiments, device 600 does not display a notification in response to determining that a set of criteria is not satisfied (e.g., when the one or more criteria and/or thresholds described in greater detail with respect to FIGS. 8, 9A, and 9B are not satisfied).

At FIG. 6S, device 600 displays notification 648 such that notification 648 overlaps a portion (e.g., some but not all and/or all) of locked user interface 650 while device 600 is in a locked state. In some embodiments, device 600 displays notification 648 in response to determining that a set of criteria is satisfied (e.g., when the one or more criteria and/or thresholds described in greater detail with respect to FIGS. 8, 9A, and 9B are satisfied). Notification 648 includes information that a threshold breathing disturbance level is satisfied (e.g., “possible sleep apnea”). In some embodiments, notification 648 includes information for which threshold breathing disturbance level is satisfied (e.g., “there is a possibility of a medium level of sleep apnea” and/or “there is a possibility of a high level of sleep apnea”). In some embodiments, device 600 detects input 605s (e.g., a touch input, an air gesture, a button press, a gaze, and/or a speech input) directed toward notification 648. In such embodiments, in response to detecting input 605s, device 600 displays an indication to unlock device 600. In some embodiments, after detecting one or more user inputs to unlock device 600 (e.g., an input corresponding to a passcode and/or an input corresponding to providing biometric information), device 600 displays an interface that includes information about the detected breathing disturbances (e.g., interface 620 of FIGS. 6E-6N, interface 602 of FIG. 6O, and/or interface 656 of FIG. 6V). In some embodiments, displaying notification 648 while device 600 is locked provides the user with timely information regarding his or her breathing disturbances while device 600 is locked.

At FIG. 6T, device 600 displays home screen user interface 652 while device 600 is in an unlocked state. In some embodiments, while in an unlocked state, device 600 is powered on and operational and is not prevented from performing at least a portion of the predefined set of operations that cannot be performed while in the locked state. Home screen user interface 652 includes application icons 654a, 654b, 654c, 654d, and 654e that, when selected, initiate the display of a respective application (e.g., a watch application, a camera application, a weather application, clock application, and/or a health application) corresponding to the selected application icon. For example, device 600 detects input 605t (e.g., a touch input, an air gesture, a button press, a gaze, and/or a speech input) directed at application icon 654e. In some embodiments, in response to detecting input 605t, device 600 displays a user interface of a health application (e.g., interface 620 of FIGS. 6E-6N, interface 602 of FIG. 6O, and/or interface 656 of FIG. 6V). In some embodiments, the health application corresponding to application icon 654c is the application that displays breathing disturbance information described herein.

At FIG. 6T, home screen user interface 652 does not include a notification, such as notification 648 of FIG. 6U. At FIG. 6T, in some embodiments, device 600 does not display a notification in response to determining that a set of criteria is not satisfied (e.g., when the one or more criteria and/or thresholds described in greater detail with respect to FIGS. 8, 9A, and 9B are not satisfied).

At FIG. 6U, device 600 displays notification 648 such that notification 648 overlaps a portion (e.g., some but not all and/or all) of home screen user interface 652 while device 600 is in an unlocked state. In some embodiments, device 600 displays notification 648 in response to determining that a set of criteria is satisfied, as described herein. In some embodiments, displaying notification 648 while device 600 displays home screen user interface 652 provides the user with timely information regarding his or her breathing disturbances while not displaying breathing disturbance data and/or a user interface of an application that is monitoring the breathing disturbance data (e.g., the health application corresponding to application icon 654c). In some embodiments, device 600 detects input 605u (e.g., a touch input, an air gesture, a button press, a gaze, and/or a speech input) directed toward notification 648. In such embodiments, in response to detecting input 605u, device 600 displays an interface that includes information about detected breathing disturbances (e.g., interface 620 of FIGS. 6E-6N, interface 602 of FIG. 6O, and/or interface 656 of FIG. 6V). In some embodiments, in response to detecting input 605u, device 600 stops displaying home screen user interface 652.

At FIG. 6V, device 600 displays breathing disturbance interface 656. In some embodiments, breathing disturbance interface 656 includes additional information and/or selectable objects that are not displayed in notification 648. Breathing disturbance interface 656 includes an indication of a level of breathing disturbance (e.g., “moderate” and/or “severe”). Breathing disturbance interface 656 also includes an indication of a period of time (e.g., “Oct. 1-31, 2023”) that corresponds to notification 648 and/or the period of time in which a set of criteria is satisfied (e.g., the period of time that the criteria and/or thresholds described in greater detail with respect to FIGS. 8, 9A, and 9B are satisfied). Additionally, breathing disturbance interface 656 includes an indication to discuss breathing disturbances with a medical professional and/or caregiver. Breathing disturbance interface 656 further includes export option 640, which is described in greater detail with respect to FIG. 6O. Breathing disturbance interface 656 also includes additional breathing disturbance information option 658 that, when selected, causes device 600 to display a user interface including additional information about breathing disturbances.

At FIG. 6V, device 600 detects input 605u (e.g., a touch input, an air gesture, a button press, a gaze, and/or a speech input) directed at done option 660. In response to detecting input 605u, device 600 stops displaying breathing disturbance interface 656 and displays a user interface (e.g., interface 620 of FIGS. 6E-6N and/or interface 602 of FIG. 6O) different from breathing disturbance interface 656. Although the description of FIGS. 6A-6V refers to device 600 and/or wearable device 606 making certain determinations, those determinations can alternatively or additionally be made by other devices, such as by a remote device and/or a server.

FIG. 7 is a flow diagram illustrating a method for controlling display of respiratory disturbances using a computer system, in accordance with some embodiments. Method 700 is performed at a computer system (e.g., 100, 300, 500, 600, and/or 606) (e.g., a smartphone, a tablet computer, a laptop computer, a desktop computer, and/or a head mounted device (e.g., a head mounted augmented reality and/or extended reality device)) that is in communication with a display generation component (e.g., 601 and/or a display generation component of 606) (e.g., a display controller, a touch-sensitive display system, a monitor, and/or a head mounted display system) and one or more input devices (e.g., 601 and/or an input device of 606) (e.g., a touch-sensitive surface, a keyboard, a controller, and/or a mouse). In some embodiments, the computer system is in communication with one or more cameras (e.g., an infrared camera, a depth camera, and/or a visible light camera). Some operations in method 700 are, optionally, combined, the orders of some operations are, optionally, changed, and some operations are, optionally, omitted.

As described below, method 700 provides an intuitive way for controlling display of respiratory disturbances. The method reduces the cognitive burden on a user for displaying of respiratory disturbances, thereby creating a more efficient human-machine interface. For battery-operated computing devices, enabling a user to display respiratory disturbances faster and more efficiently conserves power and increases the time between battery charges.

The computer system detects (702), via the one or more input devices, a request (e.g., 605b, 60501, and/or 605r) (e.g., tap, air gesture, utterance, and/or mouse click) to display a respiratory information user interface (e.g., 620). In response to detecting the request to display the respiratory information user interface, the computer system displays 704, via the display generation component, the respiratory information user interface. Displaying the respiratory information user interface includes the computer system displaying (706), via the display generation component, a graph of respiratory information (e.g., 624). The graph includes (708) one or more respiratory disturbances (e.g., 625a, 633a, and/or 633b) (e.g., a disruption in breathing while sleeping and/or sleep apnea). The graph includes (710) a plurality of time periods (e.g., 629a, 629b, 629c, 629d, and/or 629c) (e.g., days, weeks, months, and/or years); In some embodiments, a respective time period (e.g., a first time period and/or a second time period) of the set of one or more time periods is a thirty-day period (or, optionally, a seven-day period). Displaying (704) the respiratory information user interface includes, in accordance with a determination that a first set of one or more respiratory disturbances (e.g., the breathing disturbance information displayed in graph 624 in FIGS. 6B-6N) (e.g., that were detected to have occurred during the first time period) satisfy a set of criteria during a first time period (e.g., 629a, 629b, 629c, 629d, and/or 629e) of the plurality of time periods, the computer system displaying (712), via the display generation component, a first graphical object (e.g., 632b, 632c, 632d, and/or 632c) (e.g., icon, shape, horizontal bar, and/or visual element) at a first location (e.g., the location of 632b, 632c, 632d, and/or 632e in FIGS. 6H-6N), wherein the first location corresponds to (e.g., is based on a location of and/or dependent on) the first time period (e.g., location of 632b corresponds to 629b) (e.g., and not a second time period). In some embodiments, in accordance with the determination that the first set of one or more respiratory disturbances (e.g., that were detected to have occurred during the first time period) does not satisfy the set of criteria during the first time period, the computer system forgoes displaying the first graphical object at the first location. In some embodiments, the first location is adjacent to (e.g., above, below, right of, and/or left of) the graph (e.g., does not overlap with a region defined by both axes of the graph). Displaying (704) the respiratory information user interface includes, in accordance with a determination that a second set of one or more respiratory disturbances (e.g., the breathing disturbance information displayed in graph 624 in FIGS. 6B-6N) (e.g., different from the first set of one or more respiratory disturbances and/or that were detected to have occurred during the second time period) satisfy the set of criteria during a second time period (e.g., 629a, 629b, 629c, 629d, and/or 629e) of the plurality of time periods that is different from the first time period, the computer system displaying (714), via the display generation component, a second graphical object (e.g., 632b, 632c, 632d, and/or 632e) at a second location (e.g., the location of 632b, 632c, 632d, and/or 632e in FIGS. 6H-6N), different from (e.g., does not overlap with and/or is discrete from) the first location, wherein the second location corresponds to (e.g., is based on a location of and/or dependent on) the second time period (e.g., the location of 632 corresponds to 629e in FIG. 6K and/or 629d in FIG. 6L) (e.g., and not the first time period). In some embodiments, in accordance with the determination that the second set of one or more respiratory disturbances does not satisfy the set of criteria during the second time period, the computer system forgoes displaying the second graphical object at the second location. In some embodiments, the second graphical object is visually distinguished from the first graphical object (e.g., via a boundary and/or using distinct shapes). In some embodiments, the first graphical object does not extend into a region corresponding to the second time period (and/or the second graphical object does not extend into a region corresponding to the first time period). In some embodiments, the computer system concurrently displays the first graphical object at the first location and the second graphical object at the second location. In some embodiments, the second location is adjacent to (e.g., above, below, right of, and/or left of) the graph (e.g., does not overlap with a region defined by both axes of the graph). In some embodiments, the plurality of time periods are along an x-axis of the graph. In some embodiments, a y-axis of the graph corresponds to a frequency of respiratory disturbances that occur during a sleep cycle (e.g., when a computer system detects a user of the computer system is sleeping). In some embodiments, a value (e.g., a single value) on the y-axis is associated with a frequency of respiratory disturbances in a period of time (e.g., thirty minutes, one hour, and/or six hours) (and, optionally, during a detected sleep cycle and/or when a computer system detects a user of the computer system is sleeping). In some embodiments, the first graphical object (or, optionally, the second graphical object) is displayed adjacent to the graph (e.g., do not overlap with an area occupied the graph and/or do not overlap with the graph and/or do not overlap with the one or more respiratory disturbances of the graph). Conditionally displaying, with a graph of respiratory information including one or more respiratory disturbances and a plurality of time periods, a first graphical object at a first location, wherein the first location corresponds to the first time period and/or a second graphical object at a second location, different from the first location, wherein the second location corresponds to the second time period, based on whether a set of criteria is satisfied during the first time period or second time period, performs an operation when a set of conditions has been met without requiring further user input and improves visual feedback of respiratory disturbance information.

In some embodiments, the set of criteria includes a criterion that is satisfied when an entire duration (e.g., the entire duration of the month of August in FIGS. 6F-6G) (e.g., entire interval of time and/or full time period) of a respective time period (e.g., the first time period and/or the second time period, such as a 30-day period) is complete (e.g., 632b is not displayed until the entire duration of the month of August is complete, as depicted in FIGS. 6H-6I) (e.g., is fulfilled and/or has elapsed) (e.g., the first graphical object (and/or the second graphical object) is not displayed until the first time period (and/or second time period) is complete (e.g., all 30 days of the 30-day period have elapsed)). Conditionally displaying the first graphical object and/or the second graphical object based on a criterion that is satisfied when an entire duration of a respective time period is complete performs an operation when a set of conditions has been met without requiring further user input and improves visual feedback of respiratory disturbance information.

In some embodiments, the set of criteria includes a criterion that is satisfied when a threshold amount (e.g., non-zero, 10, 15, and/or 20) of respiratory disturbance events occur within a respective time period (e.g., 632b is displayed based on at least 15 sleep sessions have an elevated sleep disturbance, as depicted in FIGS. 6H-6I) (e.g., the first time period and/or the second time period) (e.g., when a person has at least 15 events over a 30-day period). In some embodiments, the threshold number of events is a threshold number of respiratory disturbance events that are detected while the computer system determines that a user of the computer system is sleeping. Conditionally displaying the first graphical object and/or the second graphical object based on a criterion that is satisfied when a threshold number of respiratory disturbance events occur within a respective time period performs an operation when a set of conditions has been met without requiring further user input and improves visual feedback of respiratory disturbance information.

In some embodiments, a respiratory disturbance event (e.g., 625a) (e.g., a single respiratory event) occurs when a threshold number of respiratory disturbances per interval of time (e.g., a person associated with device 600 has had a threshold amount of breathing disturbances per interval of time as described with respect to FIG. 6E) (e.g., a threshold of 5 respiratory disturbances per 1 hour and/or a threshold of 3 respiratory disturbances per 30 minutes) is detected for more than a threshold number of intervals of time (e.g., a person associated with device 600 has met the threshold amount of breathing disturbances per interval of time for a threshold number of intervals, as described with respect to FIG. 6E) (e.g., the threshold number of respiratory disturbances is met three times, four times, and/or five times) during a sleep session (e.g., each event is based on how many times a user has at least five hypopneas per hour while sleeping). In some embodiments, the computer system detects a number of respiratory disturbances per interval of time during a sleep session. In some embodiments, the computer system determines that a first number of intervals of time that the threshold number of respiratory disturbances per interval of time is satisfied (e.g., how many times or intervals 5 hypopneas an hour were detected). In such embodiments, the computer system determines whether the first number of intervals of time is more than a threshold number of intervals of time (e.g., were there 3 times or intervals that 5 hypopneas an hour were detected). In some embodiments, the computer system compares a number of intervals of time that the threshold number of respiratory disturbances per interval of time is satisfied relative to a number of intervals of time that the threshold number of respiratory disturbances per interval of time is not satisfied (e.g., how many times or intervals 5 hypopneas an hour were detected relative to how many times or intervals 5 hypopneas an hour were now detected) (e.g., the computer system determines a mode of respiratory disturbance events (and, optionally, not the average of respiratory disturbance events)). Using a threshold number of respiratory disturbances per interval of time is detected for more than a threshold number of intervals of time during a sleep session for a respiratory disturbance event improves the accuracy in determining whether a respiratory disturbance has occurred and whether a graphical object for a respiratory disturbance should be displayed.

In some embodiments, a respective time period (e.g., 629a, 629b, 629c, 629d, and/or 629c) (e.g., the first time period and/or second time period) begins at a respective time (e.g., the calendar day at which 629a, 629b, 629c, 629d, and/or 629e begin) (e.g., time of day, calendar day, and/or day of the month). In some embodiments, the first time period (e.g., and/or the second time period) is based on an interval of time (e.g., 15 days, 30 days, 1 month, and/or 6 months) that begins on the respective date. In some embodiments, the computer system detects that respiratory information is not available (e.g., there is gap in breathing disturbance information a portion of graph 624 in FIGS. 6K-6N) (e.g., is missing) for at least a portion (e.g., days or times that have passed, some but not all and/or all) of (e.g., time that has already passed) the respective time period (e.g., as described with respect to FIGS. 6K-6N) (e.g., respiratory information has not been detected and/or there is a gap in respiratory information). In some embodiments, in accordance with a determination that less than a threshold amount of respiratory information is available (e.g., as described with respect to FIGS. 6K-6N) (e.g., less than 5 calendar days of respiratory information is available, less than 15 calendar days of respiratory information is available, and/or less than 20 calendar days of respiratory information is available) for the respective time period, the respective time period is based (e.g., begins and/or starts on) on a first time (e.g., as depicted in FIG. 6K, 629e begins around the middle of October) (e.g., if too much respiratory information is missing, the respective time period is based on the first time). In some embodiments, in response to detecting the portion of the respective time period does not include respiratory information and in accordance with a determination that less than a threshold amount of respiratory information is available for the respective time period, the respective time period is based on a first time. In some embodiments, in accordance with a determination that more than the threshold amount of respiratory information is available (e.g., there is a gap in the breathing disturbance information on a portion of graph 624 in FIGS. 6K-6N) (e.g., more than 5 calendar days of respiratory information is available, more than 15 calendar days of respiratory information is available, and/or more than 20 calendar days of respiratory information is available) for the respective time period, the respective time period is based on (e.g., begins and/or starts on) a second time that is different from the first time (e.g., as depicted in FIG. 6L, 629d begins at the beginning of October) (e.g., even though some respiratory information is missing, there is enough respiratory information to base respective time period on the second time). In some embodiments, in accordance with the determination that less than the threshold amount of respiratory information is available for the respective time period, the respective time period includes an interval of time (e.g., 1 week, 4 weeks, and/or 1 month) that is based (e.g., begins and/or is dependent on) on a respective time (e.g., a 30 day window is based on the first time or the second time). In some embodiments, the computer system shifts the respective time period to include (e.g., encompass and/or incorporate) the greatest amount of respiratory information that is available. In some embodiments, in accordance with the determination that less than the threshold amount of respiratory information is available for the respective time period (e.g., too much respiratory information is missing), the computer system shifts the respective time period (e.g., the 30-day window shifts when too much respiratory information is missing). In some embodiments, in accordance with the determination that more than the threshold amount of respiratory information is available during the respective time period (e.g., there is enough respiratory information), the computer system forgoes shifting the respective time period (e.g., the 30-day window is maintained and/or doesn't move). In some embodiments, in response to detecting the portion of the respective time period does not include respiratory information and in accordance with a determination that more than the threshold amount of respiratory information is available during the respective time period, the respective time period is based on a second time that is different from the first time. Detecting that the respiratory information is not available and conditionally basing the respective time period on different times based on whether a threshold amount of respiratory information is available for the respective time period, performs an operation when a set of conditions has been met without requiring further user input and improves how missing respiratory information is handled by the computer system.

In some embodiments, in accordance with the determination that less than a threshold amount of respiratory information is available for the respective time period (e.g., there is a gap in breathing disturbance information on a portion of graph 624 in FIGS. 6K-6N) (and, optionally, in response to detecting that respiratory information is not available for at least a portion of the respective time period), the computer system displays, via the display generation component, a respective graphical object (e.g., the first graphical object and/or the second graphical object) at a location that corresponds to (e.g., is based on) the first time (e.g., as depicted in FIG. 6K, 632c begins around the middle of October). In accordance with a determination that more than the threshold amount of respiratory information is available for the respective time period (e.g., there is a gap in the breathing disturbance information on a portion of graph 624 in FIGS. 6K-6N), the computer system displays, via the display generation component, the respective graphical object at a location that corresponds to (e.g., is based on) the second time (e.g., as depicted in FIG. 6L, 632e begins at the beginning of October), wherein the location that corresponds to the first time is different from the location that corresponds to the second time. In some embodiments, the respective graphical object is displayed at a location that is independent of the first time and/or second time (e.g., the graphical object does not shift when a 30-day window shifts due to missing a threshold amount of missing respiratory information). Conditionally displaying a respective graphical object at a location that corresponds the first time or the second time based on whether than the threshold amount of respiratory information is available for the respective time period performs an operation when a set of conditions has been met without requiring further user input, improves how graphical objects are displayed based on missing respiratory information, and/or provides visual feedback about the missing respiratory information.

In some embodiments, the computer system displays, via the display generation component and concurrently with the first graphical object at the first location (and/or the second graphical object at the second location) (e.g., 632b, 632c, 632d, and/or 632c), a graphical element (e.g., as part of the respiratory information user interface) indicating a level of respiratory disturbances (e.g., 630a and/or 630b) (e.g., non-numeric level (e.g., low, medium, and/or high) and/or numeric level (e.g., 1, 2, and/or 5)) (e.g., elevated occurrence and/or high number of occurrences). In some embodiments, the level of respiratory disturbances indicates a level of respiratory disturbance events. In some embodiments, while concurrently displaying the first graphical object at the first location and the graphical element indicating the level of respiratory disturbances (e.g., as depicted in FIGS. 6I-6L), the computer system detects, via the one or more input devices, an input (e.g., 60511) (e.g., a touch input, an air gesture, a mouse click, a gaze, and/or a speech input) directed at the first graphical object (e.g., as depicted in FIG. 6L) (and/or the second graphical object). In some embodiments, in response to detecting the input directed at the first graphical object, displaying, via the display generation component, a graphical element indicating a possibility of sleep apnea (e.g., 634) (e.g., possible sleep apnea and/or potential sleep apnea) and/or sleep hypopnea (e.g., possible sleep hypopnea and/or potential sleep hypopnea) without displaying the graphical element indicating the level of occurrences of respiratory disturbances (e.g., as depicted in FIG. 6M). Displaying a graphical element indicating a possibility of sleep apnea without displaying the graphical element indicating the level of respiratory disturbances in response to an input directed at the first graphical object when a graphical element indicating the level of sleep disturbances is displayed, provides additional information about the graphical object and declutters the user interface.

In some embodiments, in accordance with a determination that a respective set of one or more respiratory disturbances (e.g., 625a, 625b, 633a, and/or 633b) (e.g., the first set of one or more respiratory disturbances and/or the second set of one or more respiratory disturbances) satisfy the set of criteria during a respective time period (e.g., a seven day period, 629a, 629b, 629c, 629d, and/or 629c) (e.g., the first time period and/or the second time period), the computer system initiates a communication of a notification (e.g., 642 and/or 648) (e.g., at the computer system and/or to an external computer system (e.g., smart watch, smart phone, and/or laptop) that is different from the computer system). In some embodiments, the notification is based on (and/or corresponds to) the respective set of one or more respiratory disturbances (e.g., 642 and/or 648). In some embodiments, in accordance with the determination that the respective set of one or more respiratory disturbances does not satisfy the set of criteria during the respective time period, forgoing initiation of the communication of the notification that includes information corresponding to the respective set of one or more respiratory disturbances. In some embodiments, the computer system initiates the communication of the notification prior to and/or after detecting the request to display the respiratory information user interface. Conditionally initiating a communication of a notification, to an external computer system (e.g., smart watch, smart phone, and/or laptop) that is different from the computer system, that includes information corresponding to the respective set of one or more respiratory disturbances based on a respective set of one or more respiratory disturbances satisfying a set of criteria during a respective time period, performs an operation when a set of conditions has been met without requiring further user input and provides improved visual feedback when a respective set of one or more respiratory disturbances satisfy a set of criteria during a respective time period.

In some embodiments, the computer system detects, via the one or more input devices, a request (e.g., 60502) (e.g., one or more inputs) to export information corresponding to the one or more respiratory disturbances (e.g., as described with respect to FIG. 6O). In some embodiments, in response to detecting the request to export the information corresponding to the one or more respiratory disturbances, the computer system initiates a process to export the information corresponding to the one or more respiratory disturbances (e.g., breathing disturbance information depicted in FIGS. 6E-6N, such as 625a, 625b, 633a, and/or 633b, is exported, as described with respect to FIG. 6O). In some embodiments, initiating the export of the information corresponding to the one or more respiratory disturbances includes exporting the respiratory information to a document (e.g., PDF and/or word processor document). In some embodiments, initiating the export of the information corresponding to the one or more respiratory disturbances includes initiating the printing of the information corresponding to the one or more respiratory disturbances. In some embodiments, the information corresponding to the one or more respiratory disturbances includes information corresponding to an apnea-hypopnea index (AHI) (e.g., the combined average number of apneas and/or hypopneas that occur per hour of sleep). In some embodiments, the information corresponding to the one or more respiratory disturbances is exported in a format that is different from the graph of respiratory information that includes the one or more respiratory disturbances. Initiating a process to export the information corresponding to the one or more respiratory disturbances in response to detecting the request to export the information corresponding to the one or more respiratory disturbances improves how information corresponding to the one or more respiratory disturbances is communicated to external devices and/or systems.

In some embodiments, the computer system detects that a subset of respiratory disturbances (e.g., a 7-day period of breathing disturbance information) (e.g., of a respective set of one or more respiratory disturbances (e.g., a subset (e.g., less than all) of the first set of one or more respiratory disturbances and/or a subset (e.g., less than all) of the second set of one or more respiratory disturbances)) satisfy a respective set of criteria during a seven day time period (e.g., as described with respect to FIGS. 6O-6R) (e.g., and/or prior to the first time period and/or second time period being completed; and/or after the first time period and/or second time period being completed). In some embodiments, in accordance with a determination that a subset of respiratory disturbances satisfy the respective set of criteria during a seven day time period (e.g., as described with respect to FIGS. 6O-6R) (e.g., and/or in response to detecting that the respective subset of one or more respiratory disturbances satisfies the set of criteria during the seven day time period), the computer system displays, via the display generation component, a notification (e.g., 638, 644, and/or 648) that is based on the subset of a respective set of one or more respiratory disturbances (e.g., as described with respect to FIGS. 6O-6R). In some embodiments, the notification includes a number of nights that have satisfied the criteria and/or that have had an elevated number of respiratory disturbances. In some embodiments, in accordance with a determination that the subset of respiratory disturbances does not satisfy the respective set of criteria during the seven-day time period, the computer system forgoes display of the notification that is based on the subset of a respective set of one or more respiratory disturbances. Displaying a notification that is based on a subset of respiratory disturbances in accordance with a determination that a subset of respiratory disturbances satisfy the respective set of criteria during a seven day time period provides visual feedback of whether a respective set of criteria has been satisfied and reduces the number of inputs needed to perform an operation.

In some embodiments, the one or more respiratory disturbances (e.g., the breathing disturbance information displayed in 624 of FIGS. 6E-6N) (e.g., displayed by the graph) (or, optionally, the respiratory information) is determined (e.g., inferred and/or estimated) based on data detected via an accelerometer (e.g., 608) (e.g., of the computer system and/or of an external device). In some embodiments, the computer system is in communication with a second computer system that is in communication with an accelerometer that detects the one or more respiratory disturbances. In some embodiments, the one or more respiratory disturbances is determined based on a sensor that detects the movement of a person. Basing the one or more respiratory disturbances on data detected via an accelerometer improves the computer system because using data detected via an accelerometer reduces the need for additional hardware components and/or additional sensors.

In some embodiments, the one or more respiratory disturbances (or, optionally, the respiratory information) is not determined based on data detected via a blood oxygen sensor (e.g., 610) (e.g., and/or one or more image sensors). In some embodiments, the one or more respiratory disturbances is not based on blood oxygen data (e.g., blood oxygen data that is determined from one or more image sensors and/or blood oxygen data that is detected via a blood oxygen sensor). Basing the one or more respiratory disturbances on data that is not based on data detected via a blood oxygen sensor improves the computer system because it reduces the need for additional hardware components and/or additional sensors.

In some embodiments, a dimension (e.g., a dimension of 632b, 632c, 632d, and/or 632c) (e.g., length, width, and/or height) (or, optionally, shape and/or size) of the first graphical object corresponds to (e.g., same as and/or is based on) a dimension of the first time period (e.g., a dimension of 629a, 629b, 629c, 629d, and/or 629c). In some embodiments, a dimension (e.g., a dimension of 632b, 632c, 632d, and/or 632c) (e.g., length, width, and/or height) (or, optionally, shape, and/or size) of the second graphical object corresponds to (e.g., same as and/or is based on) a dimension of the second time period (e.g., a dimension of 629a, 629b, 629c, 629d, and/or 629c). Displaying the first graphical object with a dimension that corresponds to a dimension of the first time period and the second graphical object with a dimension that corresponds to a dimension of second first time period provides visual feedback as to which time period satisfies the set of criteria.

Note that details of the processes described above with respect to method 700 (e.g., FIG. 7) are also applicable in an analogous manner to the steps described with respect to process 800 (e.g., FIG. 8) and/or method 1000 (e.g., FIG. 10). For example, one or more user interfaces and/or objects (e.g., alert indicator 632b) of method 700 are displayed based on criteria or one or more thresholds of process 800 and/or method 1000 being satisfied. For brevity, these details are not repeated herein.

FIG. 8 illustrates a flowchart of process 800 for initiating a notification using a computer system, in accordance with some embodiments. Process 800 is performed at a computer system (e.g., 100, 300, 500, 600, and/or 606) (e.g., a smartphone, a tablet computer, a laptop computer, a desktop computer, and/or a head mounted device (e.g., a head mounted augmented reality and/or extended reality device)). Some operations in process 800 are, optionally, combined, the orders of some operations are, optionally, changed, and some operations are, optionally, omitted.

At block 802, the computer system detects a triggering event. In some embodiments, the triggering event corresponds a request to begin tracking breathing disturbances. In some embodiments, the triggering event corresponds to a request to enable (e.g., activate and/or turn on) notifications for breathing disturbances. In some embodiments, notifications are enabled in response to the computer system detecting input 605b of FIG. 6B. In some embodiments, notifications are enabled in response to the computer system detecting input 605d of FIG. 6D.

At block 804, the computer system sets a time period (e.g., one day, seven days, thirty days, one month, and/or two months). In some embodiments, the computer system sets a time period similar to time window 629a, 629b, 629c, 629d, and/or 629e. In some embodiments, the computer system sets a time period based on the day the triggering event was detected. For example, if the triggering event was detected on a first triggering event date (e.g., day or time), the computer system sets the start of the time period as a first start date (which is optionally the same or different date than the first triggering event date). If the event was detected on a second triggering event date that is different from the first triggering event date, the computer system sets the start of the time period on a second start date that is different from the first start date. In some embodiments, the time period is a discrete period of time (e.g., one time period does not overlap with a different time period). In some embodiments, the time period is not a discrete time period (e.g., the time period is rolling time period and/or one period overlaps with a different time period). In some embodiments, the period of time is an uninterrupted period of time.

In some embodiments, the time period includes a plurality of sample windows (e.g., 12 hours, 24 hours, 1 week, and/or 2 weeks). In some embodiments, the computer system utilizes a single sleep session (e.g., only one sleep session) per sample window as described herein. In some embodiments, the computer system utilizes multiple sleep sessions (e.g., more than one sleep session) per sample window. In some embodiments, the sample windows are consecutive (e.g., sequential, uninterrupted, and/or successive) during the period of time set at block 804. In some embodiments, the time period set at block 804 is thirty days and the sample window is thirty 24-hour periods (e.g., 6 μm to 6 μm and/or 5 μm to 5 μm). In some embodiments, the time period is not a range of time that includes consecutive (e.g., sequential, uninterrupted, and/or successive) sample windows.

At block 806, the computer system determines whether the time period is complete (e.g., finished and/or has concluded). For example, in some embodiments, the computer system determines whether an end date (e.g., last day and/or last time) of the time period has occurred. In some embodiments, if the computer system determines that the time period is not complete, the computer system continues to monitor the time period until the time period is complete. In some embodiments, if the computer system determines that the time period is complete, the process 800 continues to block 808.

At block 808, the computer system determines whether there is at least a threshold amount (e.g., 2, 5, 10, and/or 25) of sleep sessions in the time period. In some embodiments, a sleep session is a range of time in which the computer system determines a person is sleeping. In some embodiments, to determine if a person is asleep, the computer system detects whether data detected via one or more sensors (e.g., accelerometer, gyroscope, microphone, and/or heart rate sensor) satisfies sleeping criteria (e.g., patterns and/or rules) (e.g., the person is still, lying down, and/or has a particular heart rate).

At block 808, in some embodiments, if the computer system determines there is at least a threshold amount of sleep sessions in the time period, process 800 moves to block 810. In some embodiments, if the computer system determines there is not a threshold amount of sleep sessions in the time period, the computer system does not initiate a breathing disturbance notification (and/or does not move to block 810 and/or block 812). In some embodiments, if the computer system determines there is not a threshold amount of sleep sessions in the time period, the computer system returns to block 804 to set a new time period, which is optionally different than the previously analyzed time period. In some embodiments, the new time period is different from the previously analyzed time period because the new time period includes at least one time that is not included in the previously analyzed time period.

At block 808, in some embodiments, the computer system determines whether there is at least threshold amount (e.g., 2, 5, 10, and/or 25) of valid sleep sessions in the time period. In some embodiments, not all of the detected sleep sessions qualify as a valid sleep session for the purposes of initiating the display of a breathing disturbance notification. In some embodiments, the computer system determines whether a particular sleep session during the time period satisfies valid sleep session criteria to determine if the particular sleep session is a valid sleep session. As described in greater detail herein, the valid sleep session criteria optionally includes a criterion for a duration of the sleep session (e.g., a sleep duration threshold) and/or a criterion for valid sleep data (e.g., a valid sleep data threshold). In some embodiments, determining whether a sleep session is a valid sleep session improves the computer system because it overcomes the technical limitations of tracking breathing disturbances using a wearable device, such as wearable device 606.

At block 808, in some embodiments, the computer system detects there are a plurality of sleep sessions during a sample window (e.g., a person has taken a nap during the afternoon and has slept at night during a 24-hour sample window). In some embodiments, the computer system selects a particular sleep session as a representative sleep session for a particular sample window (e.g., and does not use a different sleep session during the particular sample window). In some embodiments, the computer system selects the longest sleep duration (e.g., the session in which the person was asleep the longest and/or the session that includes the greatest amount of valid sleep data, as described with respect to FIGS. 9A-9B) during the sample window. In some embodiments, the computer system only uses sleep data (and/or breathing disturbances) from a selected sleep session and does not use sleep data (and/or breathing disturbances) from a sleep session that is not selected.

At block 808, in some embodiments, if a sleep duration (and/or amount of valid sleep data) for two or more sleep sessions during a single sample window are within a threshold amount of time of each other (e.g., 0 minutes, 5 minutes, 30 minutes, and/or 1 hour), the computer system selects a sleep session having the least amount of breathing disturbances. For example, a first sleep session includes a 5 hour sleep duration (and/or includes 5 hours of quality sleep data) and an average of 5 breathing disturbances an hour and a second sleep session includes a 5.25 hour sleep duration (and/or includes 5.25 hours of quality sleep data) and an average of 15 breathing disturbances per hour. Because the first and second sleep session are within a threshold amount of time of each other (e.g., the first and second sleep session are within 30 minutes of each other) and because the first sleep session has the least amount of breathing disturbances, the computer system selects the first sleep session as the representative sleep session for the sample window. In some embodiments, selecting a single sleep session as a representative sleep session for a sample window improves the computer system by more accurately determining when a breathing disturbance notification should be displayed. In some embodiments, the computer system determines whether each sleep session within the single sample window is a valid sleep session (e.g., as described with respect to FIGS. 9A-9B). In some embodiments, if two or more sleep sessions are both valid sleep sessions and have sleep durations that are within a threshold amount of time of each other, then the computer system selects the sleep session having the least amount of breathing disturbances.

At block 810, the computer system determines an amount of breathing disturbances for the sleep sessions (e.g., all sleep sessions and/or only the valid sleep sessions) during the time period. For example, the computer system determines an amount of breathing disturbances for each individual sleep session in the time period. In some embodiments, the computer system determines whether there is a breathing disturbance based on data received from one or more sensors (e.g., an accelerometer, microphone and/or blood oxygen sensor). In some embodiments, the computer system determines whether there is a breathing disturbance based on data received via the accelerometer and not based on data received via the blood oxygen sensor (and/or is not detected via a process to test blood oxygen level). In some embodiments, the computer system determines whether there is a breathing disturbance based on data received via an accelerometer and not on data from a sensor other than the accelerometer (e.g., the computer system does not rely on data from a sensor of the wearable device that is different from the accelerometer to determine if there is a sleep disturbance). In some embodiments, the computer system determines whether there is a breathing disturbance based on data received via a microphone and not on data from a sensor other than the microphone (e.g., the computer system does not rely on data from a sensor of the wearable device that is different from the microphone to determine if there is a sleep disturbance).

At block 810, the computer system determines that there is a breathing disturbance when breathing disturbance criteria (e.g., patterns and/or rules) are satisfied. In some embodiments, the computer system determines that there is a breathing disturbance if data (e.g., movement and/or motion) detected via the accelerometer satisfies breathing disturbance data criteria (e.g., rules and/or patterns). In some embodiments, the computer system determines that there is a breathing disturbance if data (e.g., detected via the accelerometer and/or via the blood oxygen sensor) indicates there is a threshold amount of time (e.g., 5 second, 10 seconds, and/or 15 seconds) that the person has had abnormal breathing (e.g., a partial breath (e.g., a shallow breath and/or hypopnea) and/or a stoppage of breathing (e.g., lapse in breathing and/or apnea)).

At block 810, in some embodiments, the computer system determines an average number of breathing disturbances per hour for a respective sleep session. In some embodiments, the computer system compares the total number of breathing disturbances with the total number of hours in the respective sleep session (e.g., total number of breathing disturbances per hour of sleep and/or total number of breathing disturbances that occur in valid sleep data per hour of valid sleep data). As explained in greater detail with respect to FIGS. 9A-9B, in some embodiments, the computer system uses a portion (e.g., less than all and/or all) of the sleep session to determine the average number of breathing disturbances per hour. As described herein, the computer system optionally uses less than all the data for the sleep session based on a movement of the wearable device exceeding a threshold (e.g., because the person is tossing or turning in his or her sleep) and/or automatically detecting that the wearable device is no longer physically associated (e.g., on and/or being worn) by the person. In some embodiments, a computer system determines whether the wearable device is no longer physically associated based on data detected via one or more sensors (e.g., heart rate sensor, accelerometer, one or more cameras, and/or temperature sensor) of the wearable device.

At block 810, in some embodiments, the computer system determines whether breathing disturbances for a particular sleep session correspond to one or more levels (e.g., non-elevated level, an elevated level, and/or highly elevated level) of breathing disturbances. In some embodiments, the computer system determines whether the average number of breathing disturbances per hour for a particular sleep session satisfies a threshold average (e.g., five breathing disturbances per hour, eleven breathing disturbances per hour, fifteen breathing disturbances per hour, and/or twenty breathing disturbances per hour). In some embodiments, if the average number of breathing disturbances per hour satisfies the threshold average, the respective sleep session is designated as a first type of sleep session (e.g., a sleep session having a high number of breathing disturbances and/or elevated level of breathing disturbances). In some embodiments, if the average number of breathing disturbances per hour does not satisfy the threshold average, the respective sleep session is designated as being a second type of sleep session (e.g., a sleep session having a low number of breathing disturbances and/or a sleep session having non-elevated level of breathing disturbances) that is different from the first type of sleep session.

At block 812, the computer system determines whether a breathing disturbance threshold for the time period is met. In some embodiments, to determine whether the breathing disturbance threshold is met, the computer system compares the number of sleep sessions of the first type (e.g., a sleep session having a high number of breathing disturbances and/or elevated level of breathing disturbances) to the total number of sleep sessions (e.g., valid sleep sessions and/or all sleep session) during the time period. In some embodiments, the total number of sleep sessions includes the first type of sleep session and the second type of sleep session (e.g., a sleep session having a low number of breathing disturbances and/or non-elevated level of breathing disturbances). In some embodiments, the breathing disturbance threshold is 25%, 50%, or 75% of the total number of sleep sessions correspond to the first type of sleep session (e.g., at least 25%, 50%, or 75% of the sleep sessions during the time period have an elevated level of breathing disturbances).

At block 814, if the breathing disturbance threshold is met, the computer system initiates (e.g., causes and/or triggers) the display of a breathing disturbance notification. In some embodiments, initiating the display of the breathing disturbance notification includes immediately displaying (or, optionally, not immediately displaying) the notification. In some embodiments, initiating the display of the breathing disturbance notification includes an instruction to display the notification in response to a triggering event (e.g., when device 600 is powered on, when device 600 is unlocked, and/or when device 600 is picked up, when a display of device 600 transitions states (e.g., dimmed to not dimmed or vice versa)). In some embodiments, the breathing disturbance notification is similar to notification 642 and/or notification 648 of FIGS. 6A-6V. In some embodiments, if the breathing disturbance threshold is not met, the computer system does not initiate the display of a breathing disturbance notification (and/or returns to block 804 to set a new time period).

Note that details of the processes described above with respect to process 800 (e.g., FIG. 8) are also applicable in an analogous manner to the steps described with respect to method 700 (e.g., FIG. 7) and/or method 1000 (e.g., FIG. 10). For example, process 800 optionally includes criteria or one or more thresholds described with respect to method 700 (e.g., FIG. 7) and/or method 1000 (e.g., FIG. 10). Additionally, the notification that is initiated for display in process 800 (e.g., FIG. 8) includes one or more user interfaces and/or objects of method 700. Additionally, one or more user interfaces and/or objects of method 700 are displayed in response to detecting a set of inputs that includes an input directed to the notification that is initiated for display in process 800 (e.g., FIG. 8). For brevity, these details are not repeated herein.

FIGS. 9A-9B illustrate graphs 900 of sleep data for sleep sessions, in accordance with some embodiments. As described with respect to block 808 of FIG. 8, in some embodiments, the computer system determines whether a sleep session is a valid sleep session. In some embodiments, a sleep session corresponds to a period of time in which the computer system determines that a person has slept. In some embodiments, a sleep session corresponds to a period of time in which the computer system determines that a person has continuously slept (e.g., has not woken up). In some embodiments, a sleep session corresponds to a period of time in which the computer system determines that a person has slept and has not been awake for at least a threshold amount of time (e.g., 1 second, 5 seconds, and/or 1 minute).

FIG. 9A illustrates sleep data 913 on graph 900, which includes a time of sleep duration along x-axis 904 and an amount of breathing disturbances along y-axis 902. In some embodiments, the computer system determines that a sleep session is a valid sleep session if a person has slept a threshold amount of time (also referred to as a sleep duration threshold) (e.g., 2.5 hours, 4.5 hours, and/or 7 hours), as illustrated by dashed line 906. For example, for a sleep duration that lasts until time t₁, the computer system determines that a person was not asleep long enough to satisfy the sleep duration threshold. As such, the sleep session ending at time t₁ would not qualify as a valid sleep session. As another example, for a sleep duration that lasts until time t₂, the computer system determines that that a person was asleep long enough to satisfy the sleep duration threshold. As such, a sleep session ending at time t₂ would qualify as a valid sleep session.

At FIG. 9A, in some embodiments, the computer system determines a sleep session is a valid sleep session if the sleep session has a threshold amount (e.g., 2.5 hours, 3.5 hours, or 4.5 hours) of valid sleep data (also referred to as a valid sleep data threshold). Solid lines 913a-1 and 913a-2 illustrate the portions of sleep data 913 that include valid sleep data. Semi-dashed lines 913b-1 and 913b-2 illustrate the portions of sleep data 913 that do not include valid sleep data. In some embodiments, valid sleep data correspond to portions of sleep data 913 in which a wearable device (e.g., wearable device 606) satisfies wearable device criteria. In some embodiments, the wearable device criteria includes a worn criterion that is satisfied when the wearable device is being worn by the person (e.g., the wearable device has not been taken off and/or momentarily lost satisfactory contact with the person's wrist, arm, and/or other body part). In some embodiments, the wearable device automatically detects whether the device is being worn by the person. In some embodiments, the computer system determines the wearable device is worn by a person when the computer system determines that the wearable device (and/or one or more sensors of the wearable device) is in contact (e.g., satisfactory contact and/or sufficient contact) with a portion of the person's body. In some embodiments, the computer system determines that the wearable device is worn by a person when the computer system determines that the one or more sensors of the wearable device are able to detect one or more measurements (e.g., physiological measurements and/or biological measurements) of the user's body and/or are able to detect one or more biological characteristics of the user's body (e.g., heart rate, breathing, and/or body temperature). In some embodiments, the wearable device criteria includes a movement criterion that is satisfied when the wearable device has not moved beyond a threshold amount (e.g., the wearable device has not moved because the person has not moved his or her wrist, arm, and/or other body part). In some embodiments, to determine if a sleep session qualifies as a valid sleep session, the computer system determines if a total amount of valid sleep data (e.g., solid lines 913a-1 and 913a-2) in a sleep session satisfies the valid sleep data threshold. In some embodiments, valid sleep data occurs in non-continuous (e.g., discrete and/or separated) portions of time, as depicted in FIG. 9A. For example, in some embodiments, the computer system combines a first set of valid sleep data from one portion of time during the sleep session and a second set of valid sleep data from a different portion of time during the sleep session, where the first set of valid sleep data is separated in time from the second set of valid sleep data by a period of time in which sleep data does not qualify as valid sleep data (e.g., because the wearable device momentarily lost satisfactory connection with the user and/or the wearable device moved beyond a threshold amount). In some embodiments, valid sleep data occurs throughout a continuous duration of time.

At FIG. 9A, in some embodiments, the valid sleep data threshold is satisfied while the sleep duration threshold is not satisfied (e.g., there is enough quality sleep data but the person has not slept long enough). In such embodiments, the sleep session is not a valid sleep session. For example, for a sleep duration that lasts until time t₁, the computer system determines that the valid sleep data threshold is met but the sleep duration threshold is not met. In such embodiments, the sleep session that lasts until time t₁ is not a valid sleep session.

At FIG. 9A, in some embodiments, if both the valid sleep data threshold and the sleep duration threshold are satisfied, the computer system determines the sleep session is a valid sleep session. For example, for a sleep duration that lasts until time t₂, the computer system determines that the valid sleep data threshold is satisfied and the sleep duration threshold is satisfied. As such, the sleep session that lasts until time t₂ is a valid sleep session and is subsequently selected as a sleep session to determine whether initiate the display of a notification and/or alert bar.

At FIG. 9B, graph 900 illustrates sleep data 913 for a different sleep session than the sleep session of FIG. 9A. At FIG. 9B, the computer system determines that the sleep session that lasts until time t₃ is not a valid sleep session. For example, for the sleep duration that lasts until time t₃, the computer system determines that the sleep session has not satisfied the sleep duration threshold. Additionally or alternatively, for the sleep session that lasts until time t₃, the computer system determines that the threshold amount of valid sleep data is not met based on determining that sleep data 913 corresponding to semi-dashed line 913b-3 is not valid sleep data. As such, the computer system determines that the sleep session that lasts until time t₃ is not a valid sleep session.

At FIG. 9B, the computer system determines that a sleep session that lasts until time t₄ is not a valid sleep session. At time t₄, the sleep duration threshold is satisfied (e.g., beyond dashed line 906) while the valid sleep data threshold is not satisfied (e.g., a person has slept long enough during a sleep session to qualify as valid sleep session but there is not enough quality sleep data). For a sleep duration that lasts until time t₄, while the sleep duration threshold is satisfied, the computer system determines that valid sleep data threshold is not met because there is an insufficient amount of valid sleep data (e.g., due to the wearable device momentarily losing satisfactory connection with the user and/or excessive movement during a portion of the sleep session). As described herein, sleep data 913 corresponding to sleep data 913b-3 does not qualify as valid sleep data. While sleep data 913 corresponding to solid line 913a-3 qualifies as valid sleep data, the amount of sleep data 913 corresponding to solid line 913a-3 for a sleep session that ends at time t₄ does not satisfy the valid sleep data threshold. As such, the computer system determines that the sleep session that lasts until time t₄ is not a valid sleep session.

At FIG. 9B, the computer system determines a sleep duration that lasts until time t₅ is a valid sleep session because both the sleep duration threshold and the valid sleep data threshold are satisfied. For example, the computer system determines the sleep duration that lasts until time t₅ satisfies the sleep duration threshold. Additionally or alternatively, the computer system determines that the threshold amount of valid sleep data is met because there is a sufficient amount of valid sleep data. For example, the amount of sleep data 913 corresponding to solid line 913a-3 for a sleep session that ends at time t₅ satisfies the valid sleep data threshold. As such, the computer system determines that the sleep session that lasts until time t₅ is a valid sleep session.

Note that details of the criteria described above with respect to FIGS. 9A-9B are also applicable in an analogous manner to the steps described with respect to method 700 (e.g., FIG. 7), process 800 (e.g., FIG. 8), and method 1000 (e.g., FIG. 10). For example, method 700, process 800, and/or method 1000 optionally includes criteria or one or more thresholds of FIGS. 9A-9B for determining whether a sleep session is a valid sleep session. Additionally, one or more user interfaces and/or objects (e.g., alert indicator 632b) of method 700, process 800, and/or method 1000 are displayed based on determining whether a sleep session is a valid sleep session. For brevity, these details are not repeated herein.

FIG. 1000 is a flow diagram illustrating a method 1000 for displaying a respiratory disturbance notification, in accordance with some embodiments. Method 1000 is performed at a computer system (e.g., 100, 300, 500, 600, and/or 606) (e.g., a smartphone, a smartwatch, a tablet computer, a laptop computer, a desktop computer, and/or a head mounted device (e.g., a head mounted augmented reality and/or extended reality device)) that is in communication with a one or more display generation components (e.g., 601 and/or a display generation component of 606) (e.g., a display controller, a touch-sensitive display system, a monitor, and/or a head mounted display system). In some embodiments, the computer system is in communication with one or more input devices (e.g., 601 and/or an input device of 606) (e.g., a touch-sensitive surface, a keyboard, a controller, and/or a mouse). In some embodiments, the computer system is in communication with one or more cameras (e.g., an infrared camera, a depth camera, and/or a visible light camera). Some operations in method 1000 are, optionally, combined, the orders of some operations are, optionally, changed, and some operations are, optionally, omitted.

As described below, method 1000 provides an intuitive way for displaying a respiratory disturbance notification. The method reduces the cognitive burden on a user for viewing respiratory disturbance information, thereby creating a more efficient human-machine interface. For battery-operated computing devices, enabling a user to display respiratory disturbances faster and more efficiently conserves power and increases the time between battery charges.

The computer system displays (1002), via the one or more display generation components, a respective user interface (e.g., 650, 652, and/or watch face of 606 in FIG. 6A) (e.g., a home screen, a system user interface, an application user interface, and/or a watch face including an indication of a current time, and/or a user interface having one or more application icons (e.g., that, when selected, open a respective application)). While displaying (1004) the respective user interface and in accordance with a determination that a set of respiratory disturbance criteria (e.g., the criteria and/or thresholds described with respect to 804, 806, 808, 810, and/or 812) is satisfied over a period of time (e.g., 629a, 629b, 629c, 629d, and/or 629c) (e.g., seven days, fourteen days, thirty days, one month, or two months), the computer system displays (1006), via the one or more display generation components, a notification (e.g., 648, 642, 644, and/or 656) (e.g., a selectable graphical object, a user interface, text, and/or image) corresponding to (e.g., based on and/or indicating) one or more respiratory disturbances (e.g., the breathing disturbances described with respect to block 810, the breathing disturbances corresponding to graph 624 in FIGS. 6B-6N, and/or the breathing disturbance corresponding to 913 of FIGS. 9A-9B) (e.g., a disruption (e.g., complete and/or partial) in breathing while sleeping, sleep apnea, and/or sleep hypopnea). In some embodiments, the set of respiratory disturbance criteria includes a set of sleep session criteria (e.g., the criteria and/or thresholds described with respect to 804, 806, 808, 810, and/or 812; and/or the criteria and/or thresholds described with respect FIGS. 9A-9B) that includes a sleep session criterion that is satisfied when at least a threshold amount (e.g., value, quantity, percentage, and/or ratio) of sleep sessions (e.g., a respective sleep session corresponds to a period of time in which a person sleeps and/or a length of time in which a computer system determines that a person is sleeping) from a set of sleep sessions (e.g., a set of sleep sessions within the time period set at block 804; a set of sleep sessions described with respect to block 808; and/or a set of sleep sessions that satisfy the criteria and/or thresholds of FIGS. 9A-9B) have at least a threshold amount (e.g., value, quantity, percentage, and/or ratio) of respiratory disturbances (e.g., as described with respect to block 810) (e.g., the set of sleep session criteria is satisfied when at least 50% of the set of sleep sessions have an high number of respiratory disturbances). In some embodiments, the computer system determines the set of sleep session criteria is satisfied when a ratio of the number of sleep sessions that have the threshold amount of respiratory disturbances compared to a number of sleep sessions that do not have the threshold amount of respiratory disturbances satisfies a threshold value (e.g., 80%, 90%, 100%) and/or compared to a total number of sleep sessions during the period of time (e.g., 20%, 30%, 40%, 50%, 60%, 70%, or 80%). In some embodiments, in accordance with the determination that the set of respiratory disturbance criteria is satisfied for at least the portion of the period of time, the computer system ceases display of the respective user interface. In some embodiments, the respective user interface is a user interface of a first application and the notification is initiated from a computer program (e.g., a second application and/or operating system) that is different from the first application. In some embodiments, the computer system displays the notification corresponding to one or more respiratory disturbances over (e.g., obscuring and/or hiding) one or more selectable objects (e.g., application icons and/or menu options). In some embodiments, the computer system displays the notification corresponding to one or more respiratory disturbances in a portion (e.g., some but not all and/or all) of the respective user interface.

While displaying (1004) the respective user interface and in accordance with a determination that the set of respiratory disturbance criteria is not satisfied for the period of time, forgoing (1008) display of the notification corresponding to the one or more respiratory disturbances (e.g., 648 is not displayed in FIGS. 6R and 6T; and/or as described with respect to block 812) (and, in some embodiments, maintaining display of the respective user interface). In some embodiments, while displaying the notification, the computer system detects, via one or more input devices, an input (e.g., a button press, an air gesture, a gaze, and/or a touch gesture) directed to the notification (e.g., the representation of the notification and/or a selectable object of the notification) and, in response, displays a user interface that is different from the notification. In some embodiments, in response to detecting the input directed to the notification, the computer system displays a user interface of an application that initiated to the notification (e.g., an input directed to the notification initiates the display of the application that initiated the notification). In some embodiments, the notification provides an indication (e.g., a numeric value and/or severity (e.g., low, medium, or high)) of the respiratory disturbances for an individual sleep session (e.g., a summary for an individual sleep session) and/or for the period of time (e.g., a summary for the week and/or month). Conditionally displaying a notification corresponding to the one or more respiratory disturbances based on whether the set of respiratory disturbance criteria is satisfied, where the set of respiratory disturbance criteria includes a sleep session criterion that is satisfied when at least a threshold amount of sleep sessions from a set of sleep sessions have at least a threshold amount of respiratory disturbances, performs an operation when a set of conditions has been met without requiring further user input, declutters the user interface, and surfaces relevant information without requiring further user input.

In some embodiments, wherein the set of respiratory disturbance criteria includes a time period criterion that is satisfied when at least a threshold number (e.g., 5, 10, 15, or 20) of sleep sessions occur within the period of time (e.g., as described with respect to block 808) (e.g., the notification corresponding the one or more respiratory disturbances is conditionally displayed based on whether the time period includes a minimum number of sleep sessions, independent of whether the sleep session criterion is satisfied). Including a time period criterion that is satisfied when at least a threshold number of sleep sessions occur in the period of time in order to conditionally display the notification corresponding the one or more respiratory disturbances performs an operation when a set of conditions has been met without requiring further user input, declutters the user interface, surfaces relevant information without requiring further user input, and improves the accuracy of when the notification corresponding to the one or more respiratory disturbances should be displayed.

In some embodiments, wherein the threshold amount of respiratory disturbances is a threshold average respiratory disturbances per hour during a sleep session (e.g., as described with respect to block 810 and block 812) (e.g., the threshold is an average of 5, 10, 11, 15 or 20 respiratory disturbances per hour over a single sleep session). In some embodiments, the average of respiratory disturbances per hour of sleep is the total number of respiratory disturbances (e.g., how many respiratory disturbances happened over the entire sleep duration) divided by the total duration of the sleep session (and/or a total duration of valid sleep data, as described with respect to FIGS. 8, 9A, and 9B). Including a threshold average of respiratory disturbances per hour during the sleep session as the threshold amount of respiratory disturbances performs an operation when a set of conditions has been met without requiring further user input, declutters the user interface, surfaces relevant information without requiring further user input, and improves the accuracy of when the notification corresponding to the one or more respiratory disturbances should be displayed.

In some embodiments, a respiratory disturbance corresponds to an event in which a user's breathing (e.g., breathing pattern and/or breathing rhythm) is disrupted (e.g., shallow breathes and/or stoppage in breathing) for at least a threshold amount of time (e.g., as described with respect to block 808) (e.g., 5 seconds, 10 seconds, or 15 seconds). Including an event in which a user's breathing is disrupted for at least a threshold amount of time as a respiratory disturbance surfaces relevant information without requiring further user input, provides visual feedback regarding the state of a user's breathing while sleeping, and improves the accuracy of when the notification corresponding to the one or more respiratory disturbances should be displayed.

In some embodiments, a respective sleep session is included in the set of valid sleep sessions based on (e.g., in accordance with a determination of and/or dependent on) a user sleeping (e.g., is in a sleeping state, is not awake, and/or is not awake for more than a threshold amount of time (e.g., 10 seconds, 30 seconds, or 1 minute)) for a threshold amount of time during the respective sleep session (e.g., as described with respect to block 808 and FIGS. 9A-9B) (e.g., a sleep session has to include a minimum amount of sleep time in order for the sleep session to be taken into account for the purposes of determining whether the sleep session includes the threshold amount of respiratory disturbances and/or whether the sleep session criterion is satisfied). In some embodiments, the respective sleep session is not included in the set of valid sleep sessions based on the user not sleeping for the threshold amount of time (e.g., does not wake up and/or does not move beyond a threshold amount). In some embodiments, the computer system determines a user is sleeping when data detected via one or more sensors (e.g., heart rate sensor, accelerometer, temperature sensor, and/or one or more cameras) satisfies sleep criteria (e.g., rules and/or patterns). Including a respective sleep session in the set of sleep sessions based on when a user is sleeping for a threshold amount of time during the respective sleep session, performs an operation when a set of conditions has been met without requiring further user input, declutters the user interface, surfaces relevant information without requiring further user input, and improves the accuracy of when the notification corresponding to the one or more respiratory disturbances should be displayed.

In some embodiments, a respective sleep session is included in the set of sleep sessions based on (e.g., in accordance with a determination of and/or dependent on) sleep data for the respective sleep session satisfying one or more data quality conditions for a threshold (e.g., 1.5 hours, 2.5 hours, 3.5 hours, 3.5 hours, or 4.5 hours) amount of time during the respective sleep session (e.g., as described with respect to block 808 and FIGS. 9A-9B) (e.g., a sleep session has to include a minimum amount of time of valid sleep data in order for the sleep session to be taken into account for the purposes of determining whether the sleep session includes the threshold amount of respiratory disturbances and/or whether the sleep session criterion is satisfied). In some embodiments, a respective sleep session is not included in the set of sleep sessions based on the sleep data for the sleep session not satisfying the one or more data quality conditions for the threshold (e.g., 1.5 hours, 2.5 hours, 3.5 hours, 3.5 hours, or 4.5 hours) amount of time. Including a respective sleep session in the set of sleep sessions based on sleep data for the sleep session satisfying one or more data quality conditions for a threshold amount of time during the respective sleep session, performs an operation when a set of conditions has been met without requiring further user input, declutters the user interface, surfaces relevant information without requiring further user input, and improves the accuracy of when the notification corresponding to the one or more respiratory disturbances should be displayed.

In some embodiments, the one or more data quality conditions includes a device orientation condition that is based on an orientation (e.g., an orientation with respect to a person (e.g., is the respective computer system being worn) and/or a change in orientation in physical space) of a respective computer system (e.g., as described with respect to block 808 and FIGS. 9A-9B) (e.g., the computer system and/or a wearable electronic device). In some embodiments, the one or more data quality conditions includes a worn condition that is satisfied when a computer system detects (e.g., automatically and/or without user input) that the respective computer system is worn by the user sleeping (e.g., via one or more sensors (accelerometer, heart rate sensor, temperature sensor, and/or one or more cameras)). In some embodiments, the one or more data quality conditions includes a movement condition that is satisfied when a computer system detects that the user (and/or a body part of the user (e.g., a portion of an arm (e.g., hand, wrist, and/or forearm) and/or a portion of a leg (e.g., foot and/or ankle))) has not moved beyond a threshold amount (e.g., 0.5 inches, 1 inch, or 5 inches). Including a device orientation condition that is based on an orientation of a respective computer system in the one or more data quality conditions allows the computer system to perform an operation when a set of conditions has been met without requiring further user input, declutters the user interface, surfaces relevant information without requiring further user input, and improves the accuracy of when the notification corresponding to the one or more respiratory disturbances should be displayed.

In some embodiments, the computer system detects (e.g., automatically and/or without user input), via a wearable device (e.g., the computer system and/or a wearable electronic device) (e.g., via one or more sensors of the wearable device), that a person is wearing the wearable device for a portion (e.g., a first portion) of a sleep session (e.g., as described with respect to block 808 and FIGS. 9A-9B; and/or 606 detects a person is wearing 606). In some embodiments, the computer system uses data (in some embodiments, the data is detected via one or more sensors of the wearable device) corresponding to the portion of the sleep session to determine a presence (e.g., an instance and/or an event) of a respiratory disturbance (e.g., as described with respect to block 808, 810, and FIGS. 9A-9B; and/or using data corresponding to the portion when 606 detects the person is wearing 606) (e.g., without using data corresponding to a portion of the sleep session in which the person is not wearing the wearable device). In some embodiments, the computer system uses the data corresponding to the portion of the sleep session in response to (or, optionally, based on) detecting that the user is wearing the wearable device for the portion of the sleep session. In some embodiments, the computer system detects, via the wearable device, that a user is not wearing the wearable device for a second portion of the sleep session and, in response, does not use data corresponding to the second portion of the sleep session to determine a presence of a respiratory disturbance. In some embodiments, the wearable device is worn on a body part (e.g., a portion of an arm (e.g., hand, wrist, and/or forearm) and/or a portion of a leg (e.g., foot and/or ankle)) of the user. In some embodiments, a computer system (e.g., the respective computer system, the computer system, a server, and/or the wearable device) automatically detects when the respective computer system is worn by a user based on data detected via one or more sensors (e.g., heart rate sensor, accelerometer, one or more cameras, and/or temperature sensor). Detecting that a user is wearing the wearable device for a portion of a sleep session and using data corresponding to the portion of the sleep session to determine a presence of a respiratory disturbance allows the computer system to perform an operation when a set of conditions has been met without requiring further user input and improves the accuracy of when the notification corresponding to the one or more respiratory disturbances should be displayed.

In some embodiments, the one or more respiratory disturbances is determined (e.g., inferred and/or estimated) based on data detected via an accelerometer (e.g., described with respect to block 810 and FIGS. 9A-9B; and/or respiratory disturbances are determined based on data detected via 608) (e.g., of the computer system and/or of an external device). In some embodiments, the computer system is in communication with a second computer system (e.g., a wearable device and/or remote computer system) that is in communication with the accelerometer that detects data that is used to determine the one or more respiratory disturbances. In some embodiments, the one or more respiratory disturbances is determined based on a sensor that detects movement of a user. Basing the one or more respiratory disturbances on data detected via an accelerometer improves the computer system because using data detected via an accelerometer reduces the need for additional hardware components and/or additional sensors.

In some embodiments, the one or more respiratory disturbances is not determined based on data detected via a blood oxygen sensor (e.g., 610) (e.g., and/or one or more image sensors). In some embodiments, the one or more respiratory disturbances is not based on blood oxygen data (e.g., blood oxygen data that is determined from one or more image sensors and/or blood oxygen data that is detected via a blood oxygen sensor). Basing the one or more respiratory disturbances on data that is not detected via a blood oxygen sensor improves the computer system because it reduces the need for additional hardware components and/or additional sensors.

In some embodiments, the one or more respiratory disturbances is determined based on data that is detected when a wearable device does not move beyond a threshold amount (e.g., as described with respect to blocks 808; as described in further detail with respect to FIGS. 9A-9B; and/or a breathing disturbance is not determined from data corresponding to a time at which 600 and/or 606 moves beyond a threshold amount and/or a range of time while 600 and/or 606 continues to move after 600 and/or 606 has moved beyond a threshold amount) (e.g., 0.1 inch, 0.5 inch, 1 inch, 5 or inches). In some embodiments, wherein the one or more respiratory disturbances is not determined based on data that is detected when the wearable device moves beyond the threshold amount. In some embodiments, the computer system detects that the wearable device does not move beyond a threshold amount. In some embodiments, after detecting that the wearable device has moved beyond a threshold amount, the computer system detects that the movement of the wearable device has decreased (e.g., stopped and/or slowed) and has not moved (e.g., again) beyond the threshold amount (e.g., the wearable device has moved to a new position and remains substantially still). In some embodiments, the one or more respiratory disturbances is determined based on data that is detected during the time that the computer system detects that the movement of the wearable device has decreased (e.g., stopped and/or slowed) and has not moved beyond the threshold amount (e.g., while in a new position). In some embodiments, the wearable device continues to be worn by the user while the wearable device moves in physical space (e.g., the wearable device moves based on a body part of the user moving but remains in substantially the same position relative to a portion of the body part of the user while the wearable device moves in physical space (e.g., the wearable device remains within a threshold distance (e.g., 0.5 inch, 1 inch, and/or 4 inches))). Basing the one or more respiratory disturbances on data that is detected when the wearable device does not move beyond a threshold amount improves the computer system because it reduces the need for additional hardware components and/or additional sensors.

In some embodiments, the period of time includes a respective sample window (e.g., 12 hours, 24 hours, or 1 week) including a plurality of sleep sessions (e.g., as described with respect to blocks 808). In some embodiments, the period of time corresponds to a range of time including continuous (e.g., uninterrupted) sample windows. In some embodiments, the one or more respiratory disturbances is determined based on data from a longest sleep session of the plurality of sleep sessions (e.g., as described with respect to block 808) (e.g., the sleep session that includes the longest duration of sleep as compared to the other sleep sessions of the plurality of sleep sessions) and is not determined based on data from a sleep session of the plurality of sleep sessions different from the longest sleep session (e.g., as described with respect to block 808) (e.g., if there are at least two sleep sessions, only the longest sleep session is used). In some embodiments, the computer system determines the longest sleep session based on determining which sleep session of the plurality of sleep sessions in a respective sample window includes the greatest duration of sleep (and/or longest duration of data satisfying one or more quality data conditions) as compared to the other sleep sessions of the respective sample window. Basing the one or more respiratory disturbances on data from a longest sleep session and is not based on data from a sleep session of the plurality of sleep sessions different from the longest sleep session, improves the computer system because it reduces the number of sleep sessions that need to be analyzed, which conserves battery life, and improves the accuracy of determining whether the notification corresponding to the one or more respiratory disturbances should be displayed.

In some embodiments, the period of time includes a respective sample window (e.g., as described with respect to block 804 and block 808) (e.g., 12 hours, 24 hours, or 1 week) including a first sleep session and a second sleep session (e.g., period of time in which the user is asleep and/or period of time in which data for a sleep session satisfies one or more quality data conditions). In some embodiments, the first sleep session includes a first length of sleep time (e.g., 3.5 hours, 4.5 hours, 5.5 hours) (e.g., period of time in which the user is asleep and/or period of time in which data for a sleep session satisfies one or more quality data conditions) and a first amount of respiratory disturbances (e.g., a total amount of respiratory disturbances and/or an average of respiratory disturbances per hour of the sleep session). In some embodiments, the second sleep session includes a second length of sleep time and a second amount of respiratory disturbances. In some embodiments, in accordance with a determination that the first length of sleep time is within a threshold amount of time as the second length of sleep time (e.g., as described with respect to block 808) (e.g., 10 minutes, 30 minutes, and/or 1 hour) (and/or prior to displaying the notification corresponding to one or more respiratory disturbances), the computer system selects a respective sleep session for the respective sample window. In some embodiments, in accordance with a determination that the first amount of respiratory disturbances is less than the second amount of respiratory disturbances, the computer system selects the first sleep session as the respective sleep session without selecting the second sleep session (e.g., as described with respect to block 808). In some embodiments, in accordance with a determination that the second amount of respiratory disturbances is less than the first amount of respiratory disturbances, the computer system selects the second sleep session as the respective sleep session without selecting the first sleep session (e.g., as described with respect to block 808) (e.g., the computer system uses the sleep session that has the least amount of respiratory disturbances for the purposes of determining whether the respiratory disturbance notification should be displayed). In some embodiments, by selecting the respective sleep session, the computer system uses the respective sleep session in a determination of whether the respective sleep session (e.g., along with other selected sleep sessions and not including a non-selected sleep session) satisfies the sleep session criterion (e.g., so as to trigger the notification corresponding to the one or more respiratory disturbances). In some embodiments, the computer system selects the sleep session including the least amount of respiratory disturbances from all of the sleep sessions of the respective sample window. In some embodiments, in accordance with a determination that the first length of sleep time is not within the threshold amount of time as the second length of sleep time, the computer system forgoes selection of a respective sleep session for the respective sample window. Conditionally selecting, for the respective sleep session, a sleep session that has the least amount of respiratory disturbances for the respective sample window when two sleep sessions include a length of sleep time that is within a threshold amount of time of each other, performs an operation when a set of conditions has been met without requiring further user input, declutters the user interface, and improves the accuracy of determining whether the notification corresponding to the one or more respiratory disturbances should be displayed.

In some embodiments, the computer system determines the one or more respiratory disturbances based on audio data detected via a microphone (e.g., an audio sensor and/or a microphone of wearable device 606). In some embodiments, the computer system determines if the audio data detected via the microphone satisfies a set of audio criteria (e.g., patterns and/or rules) indicating that a user has had a respiratory disturbance. In some embodiments, the set of audio criteria includes a breathing pattern criterion that is satisfied when audio data of a user's breathing corresponds to a breathing pattern that indicates the user has stopped breathing for a threshold amount of time and/or there is a blockage in the user's breathing. Basing the one or more respiratory disturbances on data detected via a microphone improves the computer system because using data detected via a microphone reduces the need for additional hardware components and/or additional sensors.

Note that details of the processes described above with respect to process 1000 (e.g., FIG. 10) are also applicable in an analogous manner to the steps described with respect to method 700 (e.g., FIG. 7) and/or process 800 (e.g., FIG. 8). For example, process 1000 optionally includes criteria or one or more thresholds described with respect to method 700 (e.g., FIG. 7) and/or process 800 (e.g., FIG. 8). Additionally, the notification that is initiated for display in process 1000 (e.g., FIG. 10) includes one or more user interfaces and/or objects of method 700. Additionally, one or more user interfaces and/or objects of method 700 are displayed in response to detecting a set of inputs that includes an input directed to the notification that is displayed in method 1000 (e.g., FIG. 10). For brevity, these details are not repeated herein.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery to users of indications of respiratory disturbances or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted respiratory disturbance information that is of greater interest to the user. Accordingly, use of such personal information data enables users to have calculated control of the respiratory disturbance information. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of respiratory disturbance content delivery services, the present technology can be configured to allow users to select to “opt in” or “opt out” of participation in the collection of personal information data during registration for services or anytime thereafter. In another example, users can select not to provide respiratory disturbance data for targeted content delivery services. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, respiratory disturbance content can be selected and delivered to users by inferring preferences based on non-personal information data or a bare minimum amount of personal information, such as the content being requested by the device associated with a user, other non-personal information available to the respiratory disturbance content delivery services, or publicly available information.

1-43. (canceled)

44. A computer system configured to communicate with a display generation component and one or more input devices, comprising:
one or more processors; and
memory storing one or more programs configured to be executed by the one or more processors, the one or more programs including instructions for:
detecting, via the one or more input devices, a request to display a respiratory information user interface; and
in response to detecting the request to display the respiratory information user interface, displaying, via the display generation component, the respiratory information user interface, wherein displaying the respiratory information user interface concurrently includes:
displaying, via the display generation component, a graph of respiratory information, wherein the graph includes:
one or more respiratory disturbances; and
a plurality of time periods;
in accordance with a determination that a first set of one or more respiratory disturbances satisfy a set of criteria during a first time period of the plurality of time periods, displaying, via the display generation component, a first graphical object at a first location, wherein the first location corresponds to the first time period; and
in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

45. The computer system of claim 44, wherein the set of criteria includes a criterion that is satisfied when an entire duration of a respective time period is complete.

46. The computer system of claim 44, wherein the set of criteria includes a criterion that is satisfied when a threshold amount of respiratory disturbance events occur within a respective time period.

47. The computer system of claim 46, wherein a respiratory disturbance event occurs when a threshold number of respiratory disturbances per interval of time is detected for more than a threshold number of intervals of time during a sleep session.

48. The computer system of claim 44, wherein a respective time period begins at a respective time, the one or more programs further including instructions for:
detecting that respiratory information is not available for at least a portion of the respective time period;
in accordance with a determination that less than a threshold amount of respiratory information is available for the respective time period, the respective time period is based on a first time; and
in accordance with a determination that more than the threshold amount of respiratory information is available for the respective time period, the respective time period is based on a second time that is different from the first time.

49. The computer system of claim 48, the one or more programs further including instructions for:
in accordance with the determination that less than a threshold amount of respiratory information is available for the respective time period, displaying, via the display generation component, a respective graphical object at a location that corresponds to the first time period; and
in accordance with a determination that more than the threshold amount of respiratory information is available for the respective time period, displaying, via the display generation component, the respective graphical object at a location that corresponds to the second time period, wherein the location that corresponds to the first time period is different from the location that corresponds to the second time period.

50. The computer system of claim 44, the one or more programs further including instructions for:
displaying, via the display generation component and concurrently with the first graphical object at the first location, a graphical element indicating a level of respiratory disturbances;
while concurrently displaying the first graphical object at the first location and the graphical element indicating the level of respiratory disturbances, detecting, via the one or more input devices, an input directed at the first graphical object; and
in response to detecting the input directed at the first graphical object, displaying, via the display generation component, a graphical element indicating a possibility of sleep apnea and/or sleep hypopnea without displaying the graphical element indicating the level of occurrences of respiratory disturbances.

51. The computer system of claim 44, the one or more programs further including instructions for:
in accordance with a determination that a respective set of one or more respiratory disturbances satisfy the set of criteria during a respective time period, initiating a communication of a notification, wherein the notification is based on the respective set of one or more respiratory disturbances.

52. The computer system of claim 44, the one or more programs further including instructions for:
detecting, via the one or more input devices, a request to export information corresponding to the one or more respiratory disturbances; and
in response to detecting the request to export information corresponding to the one or more respiratory disturbances, initiating a process to export information corresponding to the one or more respiratory disturbances.

53. The computer system of claim 44, the one or more programs further including instructions for:
detecting that a subset of respiratory disturbances satisfy a respective set of criteria during a seven day time period; and
in accordance with a determination that a subset of respiratory disturbances satisfy the respective set of criteria during a seven day time period, displaying, via the display generation component, a notification that is based on the subset of a respective set of one or more respiratory disturbances.

54. The computer system of claim 44, wherein the one or more respiratory disturbances is determined based on data detected via an accelerometer.

55. The computer system of claim 44, wherein the one or more respiratory disturbances is not determined based on data detected via a blood oxygen sensor.

56. The computer system of claim 44, wherein:
a dimension of the first graphical object corresponds to a dimension of the first time period; and
a dimension of the second graphical object corresponds to a dimension of the second time period.

57. A non-transitory computer-readable storage medium storing one or more programs configured to be executed by one or more processors of a computer system that is in communication with a display generation component and one or more input devices, the one or more programs including instructions for:
detecting, via the one or more input devices, a request to display a respiratory information user interface; and
in response to detecting the request to display the respiratory information user interface, displaying, via the display generation component, the respiratory information user interface, wherein displaying the respiratory information user interface concurrently includes:
displaying, via the display generation component, a graph of respiratory information, wherein the graph includes:
one or more respiratory disturbances; and
a plurality of time periods;
in accordance with a determination that a first set of one or more respiratory disturbances satisfy a set of criteria during a first time period of the plurality of time periods, displaying, via the display generation component, a first graphical object at a first location, wherein the first location corresponds to the first time period; and
in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.

58. A method, comprising:
at a computer system that is in communication with a display generation component and one or more input devices:
detecting, via the one or more input devices, a request to display a respiratory information user interface; and
in response to detecting the request to display the respiratory information user interface, displaying, via the display generation component, the respiratory information user interface, wherein displaying the respiratory information user interface concurrently includes:
displaying, via the display generation component, a graph of respiratory information, wherein the graph includes:
one or more respiratory disturbances; and
a plurality of time periods;
in accordance with a determination that a first set of one or more respiratory disturbances satisfy a set of criteria during a first time period of the plurality of time periods, displaying, via the display generation component, a first graphical object at a first location, wherein the first location corresponds to the first time period; and
in accordance with a determination that a second set of one or more respiratory disturbances satisfy the set of criteria during a second time period of the plurality of time periods that is different from the first time period, displaying, via the display generation component, a second graphical object at a second location, different from the first location, wherein the second location corresponds to the second time period.
